(19) United States
(12) Patent Application Publication
(10) Pub. No.: US 2008/0247975 A1
(43) Pub. Date: Oct. 9, 2008

(54) Sunscreen and personal care compositions comprising a select copolymer

(76) Inventors: Olga V. Dueva-Koganov, White Plains, NY (US); Bingham S. Jaynes, New City, NY (US); Joseph A. Lupia, Monroe, NY (US); Marcelles Van der Sluis, CL Groningen (NL); Marleen Suurmeijer, AL Joure (NL); David Normington, Leeds (GB); Marcel Schnyder, Basel (CH); Arjan Thomas Termaten, GD Assen (NL)

Correspondence Address: JoAnn Villamizar;Ciba Corporation/Patent Department, 540 White Plains Road, P.O. Box 2005, Tarrytown, NY, 10591, US

(21) Appl. No.: 12/079,606

(22) Filed: Mar. 27, 2008

Related U.S. Application Data
(60) Provisional application No. 60/922,023, filed on Apr. 5, 2007.

(51) Int. Cl. A61K 8/49; A61Q 17/04; A61Q 5/00

(52) U.S. Cl. 424/59; 424/70.9

(57) ABSTRACT

Topically applicable, water-resistant cosmetic or dermatological compositions well suited for the UV-photoprotection of human skin and/or hair comprising an effective UV— photoprotecting amount of:

(a) at least one UV screening agent;
(b) at least one select copolymer; and, optionally
(c) other cosmetically acceptable ingredients.

The present invention also relates to personal care compositions comprising at least one select copolymer and other cosmetically acceptable ingredients.

This application claims benefit under 35 USC 119(e) of U.S. Provisional app. No. 60/922,023, filed on Apr. 5, 2007, which is incorporated herein by reference.

### FIELD OF THE INVENTION

Topically applicable, water-resistant cosmetic or dermatological compositions well suited for the UV-photoprotection of human skin and/or hair comprising an effective UV-photoprotecting amount of: (a) at least one UV screening agent; and, (b) at least one select copolymer; and, optionally, (c) other cosmetically acceptable ingredients.

### BACKGROUND OF THE INVENTION

Sunscreen compositions are applied to the skin to protect the skin from the sun's ultraviolet rays that can lead to erythema, a reddening of the skin also known as sunburn. Sunlight or ultraviolet radiation in the UV-B range has a wavelength of 290 nm to 320 nm and is known to be the primary cause of sunburn. Ultraviolet rays at a wavelength of 320 nm to 400 nm, known as UV-A radiation, produces tanning of the skin. However, in the process of doing so, the UV-A rays can damage or harm the skin.

Besides the immediate malady of sunburn, excessive sunlight exposure can lead to skin disorders. For instance, prolonged and constant exposure to the sun may lead to actinic keratoses and carcinomas. Another long-term effect is premature aging of the skin. This condition is characterized by skin that is wrinkled, cracked and has lost its elasticity.

As stated above, sunscreens are typically formulated with the goal of inhibiting skin damage from the sun's rays. The sunscreen composition filters or blocks the harmful UV-A and UV-B rays that can damage and harm the skin. It is believed that sunscreen agents accomplish this by absorbing the UV-A and/or UV-B rays.

Typically, the above-described UV-B filters are combined with the above-described UV-A filters in a solution with other lipophilic or oily ingredients and solvents to form an oil phase. The solvents are used to dissolve the sunscreen actives into the oil phase. Typically, but not necessarily, the oil phase is dispersed with the help of emulsifiers and stabilizers into an aqueous solution composed primarily of water, to make an emulsion, which becomes the final sunscreen composition.

A wide variety of cosmetic compositions intended for the photoprotection (UV-A and/or UV-B) of the skin are also known to this art.

U.S. Pat. No. 5,204,090 discloses waterproof sunscreens comprising a water insoluble film forming polymer, which is incorporated herein by reference.

U.S. Pat. No. 5,653,965 discloses film forming polymers for a sunscreen spray, which is incorporated herein by reference.

U.S. Pat. No. 5,487,886 discloses acrylic polymers for sunscreen formulations, which is incorporated herein by reference.

U.S. Pat. No. 5,145,669 discloses water proof sunscreens containing crosslinked copolymer of maleic anhydride, which is incorporated herein by reference.

U.S. Pat. No. 4,663,157 discloses a copolymer of ethylene and acrylic acid for use in sunscreen compositions, which is incorporated herein by reference.

US 2006/0008427 discloses a photo-protective composition that contains a synergistic combination of a least one sunscreen agent and at least one caroteniod, which is incorporated herein by reference.

U.S. Pat. No. 7,108,860 discloses a cosmetic composition that contains at least two rheology modifying agents, which is incorporated herein by reference.

U.S. Pat. No. 7,014,842 discloses a sunscreen composition comprising one or more photoactive compounds and one or more optimization agents, which is incorporated herein by reference.

U.S. Pat. No. 6,409,998 discloses a UV-photoprotecting emulsion comprising micronized insoluble screening agents and associative polymers, which is incorporated herein by reference.

US 2004/0126339 discloses a sunscreen composition that includes a mixture of a skin bonding polymer and at least one sunscreen active ingredient, which is incorporated herein by reference.

U.S. Pat. No. 6,312,672 discloses waterproof sunscreen compositions which include polymers of isoprene, butadiene, and/or styrene, which is incorporated herein by reference.

US 2004/0091434 discloses a topically applicable photostable sunscreen composition containing at least one dibenzoylmethane UV-sunscreen and an effective amount of at least one amphiphilic block copolymer, which is incorporated herein by reference.

US 2003/0021847 discloses a composition for retaining active ingredients in personal care compositions based on one or more polymers having a network structure in an oil phase, which is incorporated herein by reference.

US 2002/0076390 discloses a composition for nails, skin and hair in the form of an aqueous emulsion or dispersion, which is incorporated herein by reference.

U.S. Pat. No. 5,688,858 discloses a polymer suitable as a dispersant, which is incorporated herein by reference.

US 2006/0104923 discloses a sunscreen composition containing fluorinated alkyl ethers, which is incorporated herein by reference.

These anti-sun or sunscreen compositions are quite often provided in the form of an emulsion, of the oil-in-water (O/W) type (namely, a cosmetically and/or dermatologically acceptable carrier comprising an aqueous dispersing continuous phase and a fatty dispersed discontinuous phase) or of the water-in-oil (W/O) type (dispersed aqueous phase in a continuous fatty phase), which contains, at various concentrations, one or more lipophilic conventional organic UV-screening agents and/or inorganic nanopigments of metal oxides, which are suited for selectively absorbing the harmful UV radiation, these screening agents (and the quantities thereof) being selected according to the desired sun protection factor (the sun protection factor (SPF) being mathematically expressed by the ratio of the irradiation time required to attain the erythematogenic threshold with the UV-screening agent to the time required to attain the erythematogenic threshold in the absence of UV-screening agent). In such emulsions, the hydrophilic screening agents are present in the aqueous phase and the lipophilic screening agents are present in the fatty phase.

The oil-in-water emulsions are, in general, more accepted by the consumer than the water-in-oil emulsions because, in particular, of their pleasant feel (similar to water) and their presentation in the form of a non-oily cream or milk; however, they also more readily lose their UV protection efficacy as soon as they come into contact with water. Indeed, the hydrophilic screening agents tend to disappear in water, upon washing in the sea or in a swimming pool, under the shower or when engaged in water sports; thus, anti-sun or sunscreen compositions containing same, whether alone or combined with lipophilic screening agents, no longer provide the desired initial protection as soon as the substrate (skin or hair) to which they have been applied is contacted with water.

Anti-sun (sunscreen) compositions exhibiting improved resistance to water have been formulated as water-in-oil emulsions. Indeed, a hydrophilic screening agent is more stable to water in a water-in-oil emulsion than in an oil-in-water emulsion. However, as indicated above, such compositions are not yet completely satisfactory since they promote, after application, a fat-like impression which is particularly unpleasant for the user.

Thus, serious need continues to exist for anti-sun or sunscreen compositions which impart to the skin and/or the hair effective solar protection which is stable over time and resistant to water (stability to water) and the cosmetic performance of which presents features that would be comparable to those obtained with conventional oil/water emulsions.

### SUMMARY OF THE INVENTION

It is now surprisingly and unexpectedly determined that specific sunscreen compositions containing at least one UV-screening agent and at least one select copolymer not only provide anti-sun compositions whose cosmetic performance features are comparable to those generally associated with a conventional sunscreen composition formulated as an oil/water emulsion, but also exhibit good stability as well as enhanced stability to water.

Therefore, a first aspect of the present invention is directed to a sunscreen composition comprising at least one sunscreen agent, at least one select copolymer of formula (I), and other cosmetically acceptable ingredients.

A second aspect of the present invention is directed to a method of preparing a sunscreen composition comprising mixing together at least one sunscreen agent, at least one select copolymer of formula (I) and, optionally, other cosmetically acceptable ingredients.

A third aspect of the present invention is directed to a method of increasing the sun protection factor of a sunscreen composition wherein said method comprises incorporating into said compositions an effective amount of at least one select copolymer according to formula (I).

A fourth aspect of the present invention is directed to a method of improved UV protection of mammalian hair and/or skin from the damaging effects of UV radiation wherein said method comprises applying to said skin and/or said hair an effective amount of a sunscreen composition comprising at least one sunscreen agent, at least one select copolymer of formula (I), and, optionally, other cosmetically acceptable ingredients.

A fifth aspect of the present invention is directed to a cosmetic or dermatological composition comprising a select copolymer of formula (I) and other cosmetically acceptable ingredients.

### DETAILED DESCRIPTION OF THE INVENTION

The present invention provides a sunscreen composition comprising:

(a) at least one UV screening agent;
(b) at least one select copolymer comprising

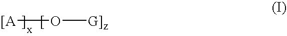

wherein
x and z represent the percentage by weight that each repeating unit or derived monomer is contained within the copolymer;
x and z refer to repeating units;
x and z add up to total 100 weight percent relative to the total weight of the copolymer;
z is from about 0.001% to about 99.999% by weight of the copolymer;
x is from about 0.001% to about 99.999% by weight of the copolymer;
A is a polymer;
G is covalently bonded to the polymer A through an oxygen linking group;
O is an oxygen atom;

### G is

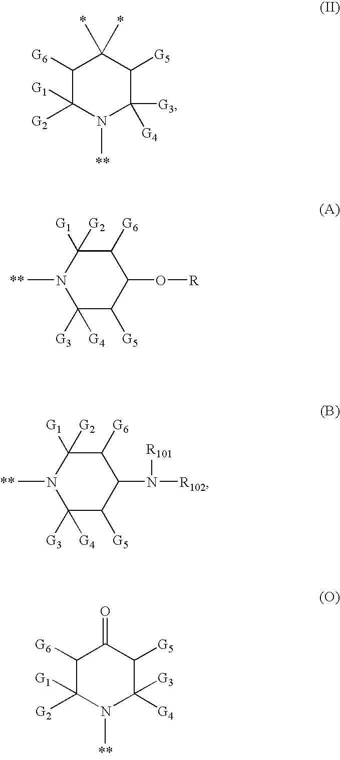

wherein
G₁, G₂, G₃, G₄ are independently C₁-C₆alkyl or G₁ and G₂ or G₃ and G₄, or G₁ and G₂ and G₃ and G₄ together form a C₅-C₁₂cycloalkyl group;
G₅, G₆ independently are H, C₁-C₁₈alkyl, phenyl, naphthyl or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, of a cycloaliphatic carboxylic acid having 7 to 15 carbon atoms, or an alpha, beta-unsaturated carboxylic acid having 3 to 5 carbon atoms or of an aromatic carboxylic acid having 7 to 15 carbon atoms;
R₁₀₁ is C₁-C₁₂alkyl, C₅-C₇cycloalkyl, C₇-C₈aralkyl, C₂-C₁₈alkanoyl, C₃-C₅alkenoyl or benzoyl;
R₁₀₂ is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or

### G is

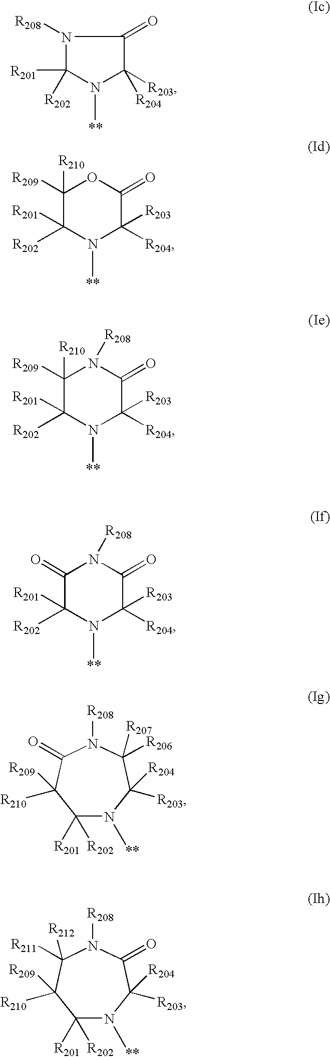

wherein R₂₀₁, R₂₀₂, R₂₀₃ and R₂₀₄ independently of each other are C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl or R₂₀, and R₂₀₂ and/or R₂₀₃ and R₂₀₄ together with the linking carbon atom form a C₃-C₁₂cycloalkyl radical;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by one or more OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉phenylalkyl, C₅-C₁₀heteroaryl, —C(O)—C₁-C₁₈alkyl, —O—C₁-C₁₈alkyl or —COOC₁-C₁₈alkyl;
R₂₀₉, R₂₁₀, R₂₁₁ and R₂₁₂ are independently hydrogen, phenyl or C₁-C₁₈alkyl; or

### G is

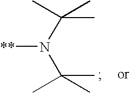

### G is

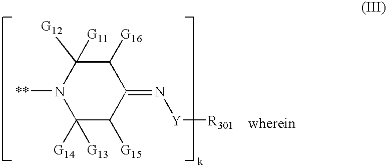

G₁₁, G₁₂, G₁₃ and G₁₄ are independently C₁-C₄alkyl or G₁₁ and G₁₂ together and G₁₃ and G₁₄ together, or G₁₁ and G₁₂ together or G₁₃ and G₁₄ together are pentamethylene;
G₁₅ and G₁₆ are each independently of the other hydrogen or C₁-C₄alkyl;
X is as defined above;
k is 1, 2, 3, or 4
Y is O or NR₃₀₂ or when k is 1 and R₃₀₁ represents alkyl or aryl Y is additionally a direct bond;
R₃₀₂ is H, C₁-C₁₈alkyl or phenyl;
if k is 1
R₃₀₁ is H, straight or branched C₁-C₁₈alkyl, C₃-C₁₈alkenyl or C₃-C₁₈alkinyl, which may be unsubstituted or substituted, by one or more OH, C₁-C₈alkoxy, carboxy, C₁-C₈alkoxycarbonyl;
C₅-C₁₂cycloalkyl or C₅-C₁₂cycloalkenyl;
phenyl, C₇-C₉phenylalkyl or naphthyl which may be unsubstituted or substituted by one or more C₁-C₈alkyl, halogen, OH, C₁-C₈alkoxy, carboxy, C₁-C₈alkoxycarbonyl;
—C(O)—C₁-C₃₆alkyl, or an acyl moiety of a □,□-unsaturated carboxylic acid having 3 to 5 carbon atoms or of an aromatic carboxylic acid having 7 to 15 carbon atoms;
—SO₃⁻Q⁺, —PO(O⁻Q⁺)₂, —P(O)(OC₁-C₈alkyl₂)₂, —P(O)(OH₂)₂, —SO₂—OH, —SO₂—C₁-C₈alkyl, —CO—NH—C₁-C₈alkyl, —CONH₂, COO—C₁-C₈alkyl₂, COOH or Si(Me)₃, wherein Q⁺ is H⁺, ammonium or an alkali metal cation;
if k is 2
R₃₀₁ is C₁-C₁₈alkylene, C₃-C₁₈alkenylene or C₃-C₁₈alkinylene, which may be unsubstituted or substituted, by one or more OH, C₁-C₈alkoxy, carboxy, C₁-C₈alkoxycarbonyl; or xylylene; or
R₃₀₁ is a bisacyl radical of an aliphatic dicarboxylic acid having 2 to 36 carbon atoms, or a cycloaliphatic or aromatic dicarboxylic acid having 8-14 carbon atoms;
if k is 3,
R₃₀₁ is a trivalent radical of an aliphatic, cycloaliphatic or aromatic tricarboxylic acid; and
if k is 4, R₃₀₁ is a tetravalent radical of an aliphatic, cycloaliphatic or aromatic tetracarboxylic acid;
and, optionally
(c) other cosmetically acceptable ingredients.

According to the instant invention, the instant copolymers of formula (I) have a structure that is selected from the group consisting of copolymer, terpolymer, block, star, random, linear, branched, crosslinked and not crosslinked.

According to the instant invention, the group “—O-G” can be reacted into the polymer “A” during synthesis of polymer A or the group “—O-G” can be reacted or grafted onto polymer “A” during processing; for example, during coextrusion.

The above compounds and their preparation are described in GB2335190, GB 2342649 and GB2361235.

The 4 imino compounds of formula III can be prepared for example according to E. G. Rozantsev, A. V. Chudinov, V. D. Sholle.:Izv. Akad. Nauk. SSSR, Ser. Khim. (9), 2114 (1980), starting from the corresponding 4-oxonitroxide in a condensation reaction with hydroxylamine and subsequent reaction of the OH group. The compounds are described WO 02/100831.

Another embodiment of the instant invention is for formula (I) G is

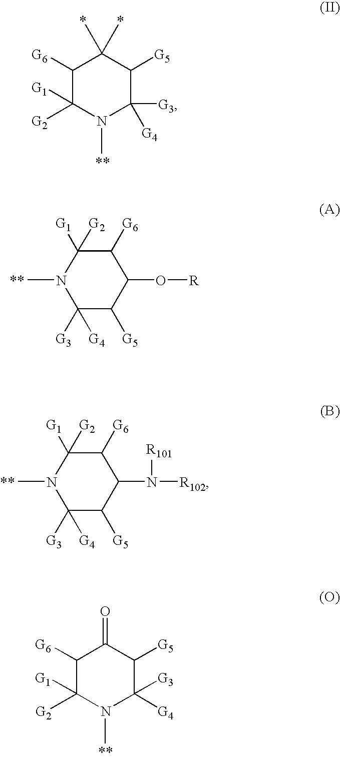

wherein
G₁, G₂, G₃, G₄ are independently C₁-C₁alkyl or G₁ and G₂ or G₃ and G₄, or G₁ and G₂ and
G₃ and G₄ together form a C₅-C₁₀cycloalkyl group;
G₅, G₆ independently are H, C₁-C₁₈alkyl, phenyl, or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, or of a cycloaliphatic carboxylic acid having 7 to 15 carbon atoms;
R₁₀₁, is C₁-C₁₂alkyl, C₅-C₇cycloalkyl, C₇-C₈aralkyl, C₂-C₁₈alkanoyl, C₃-C₅alkenoyl or benzoyl;
R₁₀₂ is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or

### G is

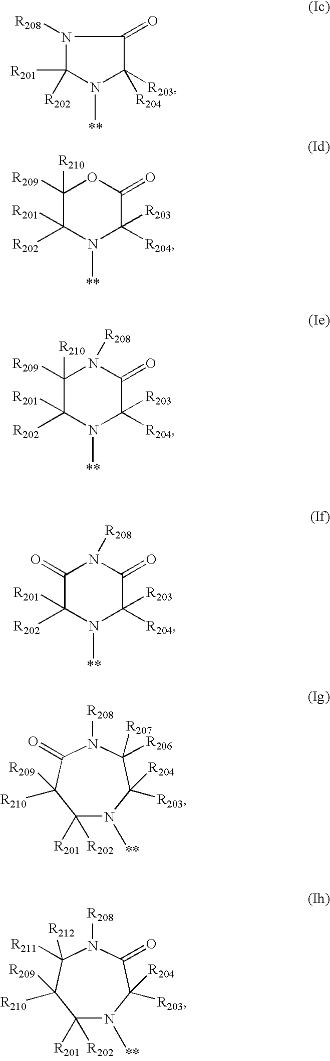

wherein R₂₀₁, R₂₀₂, R₂₀₃ and R₂₀₄ independently of each other are C₁-C₆alkyl, C₃-C₁₈alkenyl; C₁-C₁₈alkyl, or C₃-C₁₈alkenyl, which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl; C₁-C₁₈alkyl which are substituted by one or more OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉phenylalkyl, C₅-C₁₀heteroaryl, —C(O)—C₁-C₁₈alkyl, —O—C₁-C₁₈alkyl or —COOC₁-C₁₈alkyl;
R₂₀₉, R₂₁₀, R₂₁₁ and R₂₁₂ are independently hydrogen, phenyl or C₁-C₁₈alkyl; or

### G is

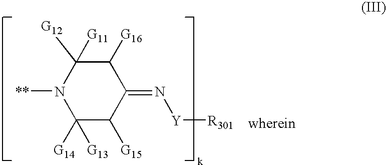

G₁₁, G₁₂, G₁₃ and G₁₄ are independently C₁-C₄alkyl or G₁₁ and G₁₂ together and G₁₃ and G₁₄ together, or G₁₁ and G₁₂ together or G₁₃ and G₁₄ together are pentamethylene;
G₁₅ and G₁₆ are each independently of the other hydrogen or C₁-C₄alkyl;
k is 1
Y is O or NR₃₀₂ or Y is additionally a direct bond;
R₃₀₂ is H, C₁-C₁₈alkyl or phenyl;
if k is 1
R₃₀₁ is H, straight or branched C₁-C₁₈alkyl, C₃-C₁₈alkenyl or C₃-C₁₈alkinyl, which may be unsubstituted or substituted, by one or more OH, C₁-C₈alkoxy, carboxy, C₁-C₈alkoxycarbonyl;
C₅-C₁₂cycloalkyl or C₅-C₁₂cycloalkenyl; C₇-C₉phenylalkyl which may be unsubstituted or substituted by one or more C₁-C₈alkyl, halogen, OH, C₁-C₈alkoxy, carboxy, C₁-C₈alkoxycarbonyl; —C(O)—C₁-C₃₆alkyl;
—SO₃⁻Q⁺, —PO(O⁻Q⁺)₂, —P(O)(OC₁-C₈alkyl₂)₂, —P(O)(OH₂)₂, —SO₂—OH, —SO₂—C₁-C₈alkyl, —CO—NH—C₁-C₈alkyl, —CONH₂, COO—C₁-C₈alkyl₂, COOH or Si(Me)₃, wherein Q₊ is H⁺, ammonium or an alkali metal cation.

Another embodiment of the instant invention is for formula (I) G is

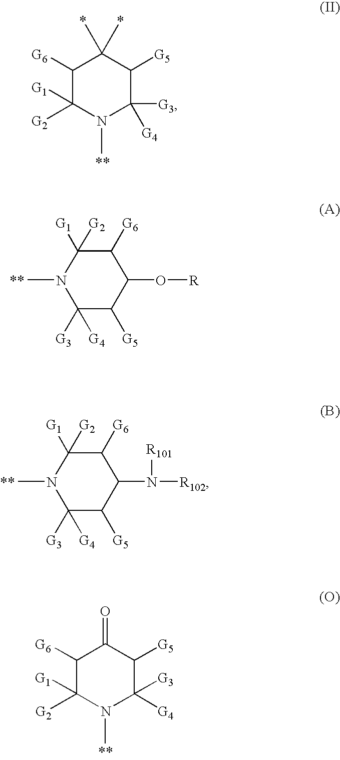

wherein
G₁, G₂, G₃, G₄ are independently C₁-C₁₋alkyl or G₁ and G₂ or G₃ and G₄, or G₁ and G₂ and G₃ and G₄ together form a C₅-C₁₀cycloalkyl group;
G₅, G₆ independently are H, C₁-C₁₈alkyl, phenyl, or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, or of a cycloaliphatic carboxylic acid having 7 to 15 carbon atoms;
R₁₀₁ is C₁-C₁₂alkyl, C₅-C₇cycloalkyl, C₇-C₈aralkyl, C₂-C₁₈alkanoyl, C₃-C₅alkenoyl or benzoyl;
   R₁₀₂ is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or

### G is

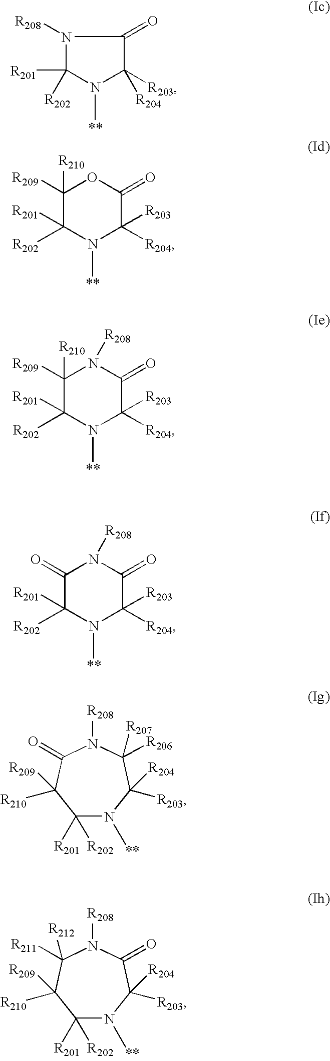

wherein R₂₀₁, R₂₀₂, R₂₀₃ and R₂₀₄ independently of each other are C₁-C₆alkyl, C₃-C₁₈alkenyl; C₁-C₁₈alkyl, or C₃-C₁₈alkenyl, which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl; C₁-C₁₈alkyl which are substituted by one or more OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉-phenylalkyl, C₅-C₁₀heteroaryl, —C(O)—C₁-C₁₈alkyl, —O—C₁-C₁₈alkyl or —COOC₁-C₁₈alkyl;
R₂₀₉, R₂₁₀, R₂₁₁ and R₂₁₂ are independently hydrogen, phenyl or C₁-C₁₈alkyl.

The alkyl radicals in the various substituents may be linear or branched. Examples of alkyl containing 1 to 18 carbon atoms are methyl, ethyl, propyl, isopropyl, butyl, 2-butyl, isobutyl, t-butyl, pentyl, 2-pentyl, hexyl, heptyl, octyl, 2-ethylhexyl, t-octyl, nonyl, decyl, undecyl, dodecyl, tridecyl, tetradecyl, hexadecyl and octadecyl.

Alkenyl with 3 to 18 carbon atoms is a linear or branched radical as for example propenyl, 2-butenyl, 3-butenyl, isobutenyl, n-2,4-pentadienyl, 3-methyl-2-butenyl, n-2-octenyl, n-2-dodecenyl, iso-dodecenyl, oleyl, n-2-octadecenyl or n-4-octadecenyl. Preferred is alkenyl with 3 to 12, particularly preferred with 3 to 6 carbon atoms.

Alkynyl with 3 to 18 is a linear or branched radical as for example propinyl

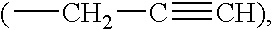

2-butinyl, 3-butinyl, n-2-octinyl, or n-2-octadecinyl. Preferred is alkinyl with 3 to 12, particularly preferred with 3 to 6 carbon atoms.

Examples for hydroxy substituted alkyl are hydroxy propyl, hydroxy butyl or hydroxy hexyl.

Examples for halogen substituted alkyl are, for example, dichloropropyl, monobromobutyl or trichlorohexyl.

C₂-C₁₈alkyl interrupted by at least one O atom is for example —CH₂—CH₂—O—CH₂—CH₃, —CH₂—CH₂—O—CH₃— or —CH₂—CH₂—O—CH₂—CH₂—CH₂—O—CH₂—CH₃—. It is preferably derived from polyethlene glycol. A general description is —((CH₂)ₐ—O)_{b}—H/CH₃, wherein a is a number from 1 to 6 and b is a number from 2 to 10.

C₂-C₁₈alkyl interrupted by at least one NR₂₀₅ group may be generally described as—((CH₂)ₐ—NR₂₀₅)_{b}—H/CH₃, wherein a, b and R₂₀₅ are as defined above.

C₃-C₁₂cycloalkyl is typically, cyclopropyl, cyclopentyl, methylcyclopentyl, dimethylcyclopentyl, cyclohexyl, methylcyclohexyl or trimethylcyclohexyl.

C₆-C₁₀ aryl is for example phenyl or naphthyl, but also comprised are C₁-C₄alkyl substituted phenyl, C₁-C₄alkoxy substituted phenyl, hydroxy, halogen or nitro substituted phenyl. Examples for alkyl substituted phenyl are ethylbenzene, toluene, xylene and its isomers, mesitylene or isopropylbenzene. Halogen substituted phenyl is for example dichlorobenzene or bromotoluene.

Alkoxy substituents are typically methoxy, ethoxy, propoxy or butoxy and their corresponding isomers.

C₇-C₉-phenylalkyl is benzyl, phenylethyl or phenylpropyl.

C₅-C₁₀heteroaryl is for example pyrrol, pyrazol, imidazol, 2, 4, dimethylpyrrol, 1-methylpyrrol, thiophene, furane, furfural, indol, cumarone, oxazol, thiazol, isoxazol, isothiazol, triazol, pyridine, alpha-picoline, pyridazine, pyrazine or pyrimidine.

If R is a monovalent radical of a carboxylic acid, it is, for example, an acetyl, propionyl, butyryl, valeroyl, caproyl, stearoyl, lauroyl, acryloyl, methacryloyl, benzoyl, cinnamoyl or beta-(3,5-di-tert-butyl-4-hydroxyphenyl)propionyl radical.

C₁-C₁₈alkanoyl is for example, formyl, propionyl, butyryl, octanoyl, dodecanoyl but preferably acetyl and C₃-C₅alkenoyl is in particular acryloyl.

UV screening agents of component (a) useful in the present invention include organic sunscreens and/or inorganic sunscreens which are preferably active in the UV-A and/or UV-B regions (UV absorbers), and are soluble in water or in fats or insoluble in, e.g., cosmetic solvents commonly used. Typically, the compositions of the present invention contain combinations of one or more sunscreen agents. Additionally, the compositions of the present invention contain combinations of two or more sunscreen agents. The combination of sunscreen agents of component (a) can be, for example: two or more inorganic sunscreen agents; two or more organic soluble sunscreen agents; two or more organic micronized or micronizable sunscreen agents; and/or mixtures thereof.

Representative inorganic sunscreens of component (a) include pigments, or alternatively nanopigments (mean size of the primary particles: generally between 5 nm and 100 nm, preferably between 10 nm and 50 nm) formed from coated or uncoated metal oxides, such as, for example, titanium oxide (amorphous or crystalline in the rutile and/or anatase form), iron oxide, zinc oxide, zirconium oxide or cerium oxide nanopigments, which are all known in the art as UV sunscreens. Conventional coating agents are, furthermore, alumina and/or aluminium stearate. Such nanopigments formed from coated or uncoated metal oxides are disclosed in particular in EP 518 772 and EP 518 773.

The organic sunscreens of component (a) may be soluble (non-micronized) or insoluble (micronized or micronizable) in the sunscreen composition of the instant invention.

Suitable insoluble (micronized or micronizable) organic sunscreens or UV absorbers of component (a) may be, e.g. a triazine, a benzotriazole, a benzophenone, a vinyl group-containing amide, a cinnamic acid amide or a sulfonated benzimidazole UV absorber.

A preferred class of triazine compounds is that having the formula

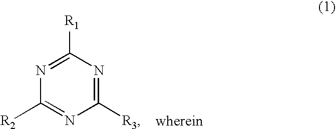

R₁, R₂ and R₃, independently from each other, are hydrogen; hydroxy; C₁-C₃alkoxy; NH₂; NHR₄⁻; N(R₄)₂; OR₄; C₆-C₁₂aryl; phenoxy; anilino; pyrrolo; in which the respective phenyl, phenoxy, anilino or pyrrolo moieties are not substituted or substituted by one, two or three substitutents selected from OH, carboxy, CO—NH₂, C₁-C₁₂alkyl, C₁-C₁₂alkoxy, a methylidenecamphor group, a group —(CH═CH)ₘC(═O)—OR₄, a group

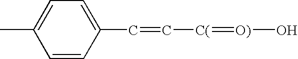

or the corresponding alkali metal, ammonium, mono-, di- or tri-C₁-C₄alkylammonium, mono-, di- or tri-C₂-C₄alkanolammonium salts, or the C₁-C₃alkyl esters thereof or by a radical of formula

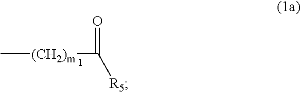

R₄ is C₁-C₅alkyl;
R₅ is hydroxy; C₁-C₅alkyl that is unsubstituted or substituted by one or more OH groups; C₁-C₅alkoxy; amino; mono- or di-C₁-C₅alkylamino; M; a radical of formula

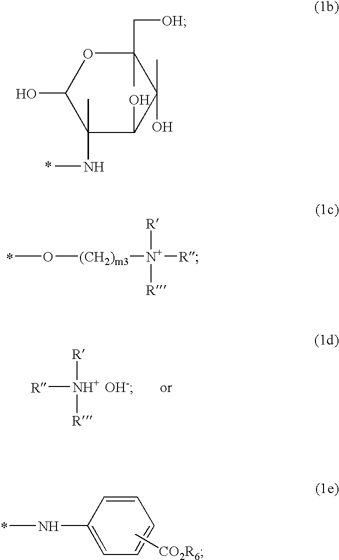

R′, R″ and R′″ independently of the other are C₁-C₁₄alkyl that is unsubstituted or substituted by one or more OH groups;
R₆ is hydrogen; M; C₁-C₅alkyl; or a radical of the formula —(CH₂)ₘ_{₂}—O-T₁;
   M is a metal cation;
   T₁ is hydrogen; or C₁-C₈alkyl;
   m is 0 or 1;
   m₁ is from 1 to 5;
   m₂ is from 1 to 4; and
   m₃ is from 2 to 14.

Preferred compounds of formula (1) are those, wherein
R₁, R₂ and R₃ independently from each other are a radical of formula

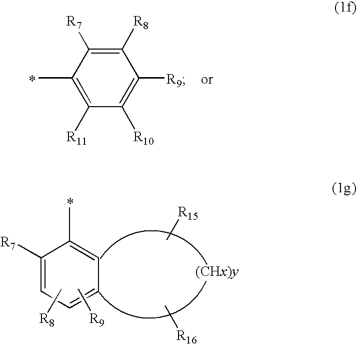

R₇ and R₁₁ independently from each other are hydrogen; C₁-C₁₈alkyl; or C₆-C₁₂aryl;
R₈, R₉ and R₁₀, independently from each other, are hydrogen; or a radical of formula

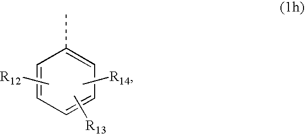

wherein, in formula (1f), at least one of the radicals R₈, R₉
and R₁₀ are a radical of formula (1h);
R₁₂, R₁₃, R₁₄, R₁₅ and R₁₆ independently from each other are hydrogen; hydroxy; halogen; C₁-C₁₈alkyl; C₁-C₁₈alkoxy; C₆-C₁₂aryl; biphenylyl; C₆-C₁₂aryloxy; C₁-C₁₈alkylthio; carboxy; —COOM; C₁-C₁₈-alkylcarboxyl; aminocarbonyl; or mono- or di-C₁-C₁₈alkylamino; C₁-C₁₀acylamino; —COOH;
   M is an alkali metal ion;
   x is 1 or 2; and
   y is a number from 2 to 10.

Most preferred triazine derivatives are compounds of formula

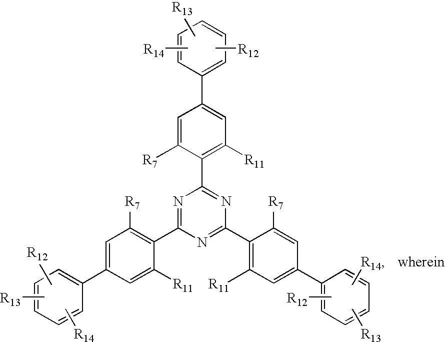

R₇, R₁₁, R₁₂, R₁₃ and R₁₄ are defined as in formula (1f), (1g) or (1h), and most preferably compound of formula (2), wherein
R₇ and R₁₁ are hydrogen.

Furthermore, triazine derivatives of formula

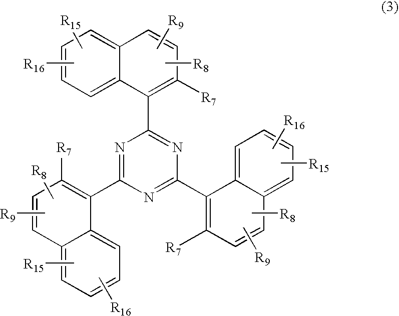

are preferred, wherein
R₇, R₈, R₉, R₁₅ and R₁₆ are defined as in formula (1g), and most preferably compounds of formula (3), wherein
R₇, R₈, R₉, R₁₅ and R₁₆ are hydrogen; or, independently from each other, C₁-C₁₈alkyl.

Most preferred as component (a) are triazine derivatives of formula

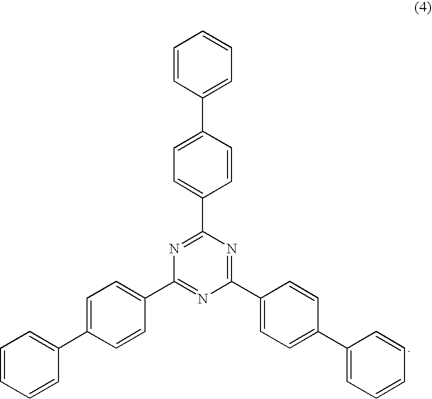

Further preferred triazine derivatives according to component (a) correspond to formula

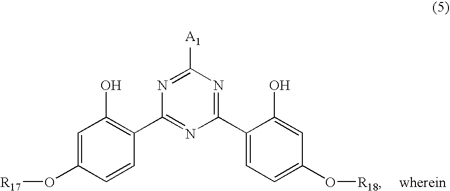

R₁₇ and R₁₈, independently of one another, are C₁-C₁₈alkyl; C₂-C₁₈alkenyl; a radical of the formula —CH₂—CH(—OH)—CH₂—O-T₁; or a radical of the
   formula

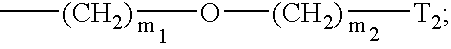

a radical of the formula

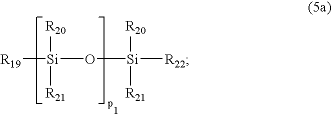

R₁₉ is a direct bond; a straight-chain or branched C₁-C₄alkylene radical or a radical of the formula

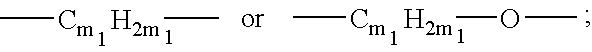

R₂₀, R₂₁ and R₂₂, independently of one another, are C₁-C₁₈alkyl; C₁-C₁₈alkoxy or a radical of the formula

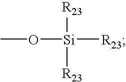

R₂₃ is C₁-C₅alkyl;
T₁ and T₂, independently from each other, are hydrogen; or C₁-C₈alkyl;
m₁, m₂ and m₃, independently of one another, are 1 to 4;
p₁ is 0; or a number from 1 to 5;
A₁ is a radical of the formula

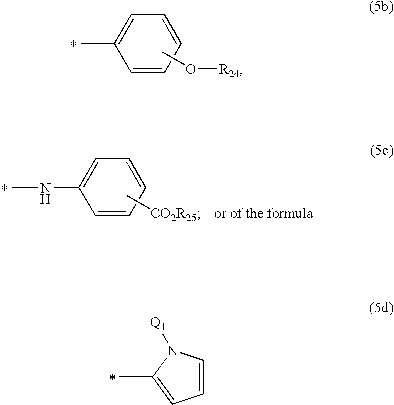

R₂₄ is hydrogen; C₁-C₁₀alkyl, —(CH₂CHR₂₆—O)ₙ_{₁}—R₂₅; a —CH₂—CH(—OH)—CH₂—O-T₁; or radical of the formula

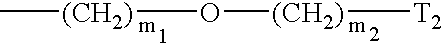

R₂₅ is hydrogen; M; C₁-C₅alkyl; or a radical of the formula —(CH₂)ₘ—O-T₁;
R₂₆ is hydrogen; or methyl;
Q₁ C₁-C₁₈alkyl;
   M is a metal cation; and
   n₁ is 1-16.

Further preferred triazine derivatives according to component are compounds of formulae

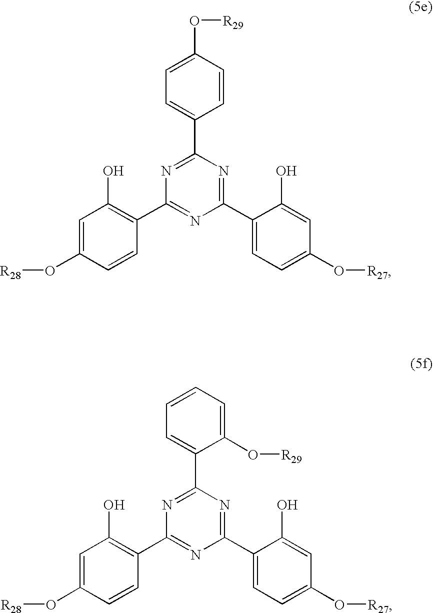

the formula

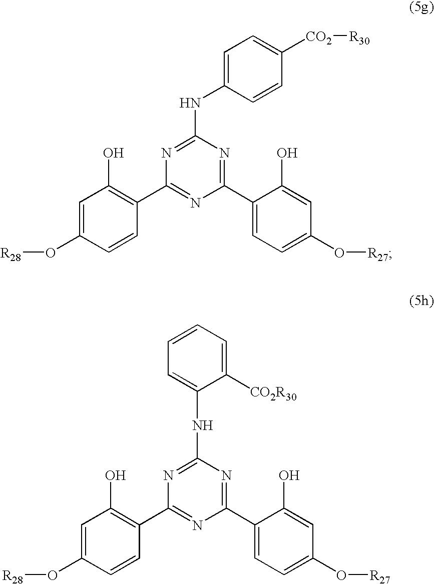

in which
R₂₇ and R₂₈, independently of one another, are C₃-C₁₈alkyl; or —CH₂—CH(—OH)—CH₂—O-T₁;
R₃₀ is C₁-C₁₀alkyl or a radical of the formula

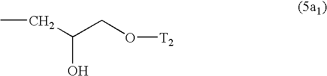

or the formula

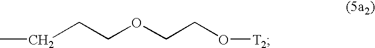

R₃₀ is hydrogen; M; C₁-C₅alkyl; —NH—C₁-C₅alkyl, preferably —NH-tert.alkyl; or a radical of the formula —(CH₂)ₘ—O-T₂;
   T₁ and T₂, independently of one another, are hydrogen; or C₁-C₅alkyl; and
   m is 1 to 4.

Uppermost of interest are compounds of the formulae (5e) and (5f), in which
R₂₇ and R₂₈, independently of one another, are C₃-C₁₈alkyl; or —CH₂—CH(—OH)—CH₂—O-T₁;
R₂₉ is C₁-C₁₀alkyl;
   and compounds of the formulae (5g) and (5h), in which
R₂₇ and R₂₈, independently of one another, are C₃-C₁₈alkyl or —CH₂—CH(—OH)—CH₂—O-T₁;
   and
   T₁ is hydrogen; or C₁-C₅alkyl.

Very particularly preferred in this case are triazine compounds of the formula (5e)-(5h), in which R₂₇ and R₂₈ have the same meaning.

Furthermore, interesting triazines correspond to the formula

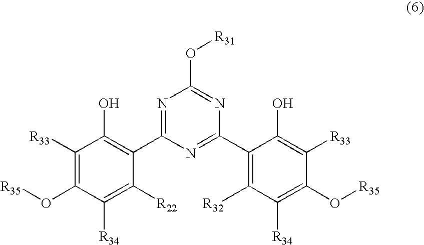

in which
R₃₁ is C₁-C₃₀alkyl; C₂-C₃₀alkenyl; unsubstituted or C₁-C₅alkyl-mono- or polysubstituted C₅-C₁₂cycloalkyl, C₁-C₅alkoxy-C₁-C₁₂alkyl; amino-C₁-C₁₂alkyl;
   C₁-C₅monoalkylamino-C₁-C₁₂alkyl; C₁-C₅dialkylamino-C₁-C₁₂alkyl; a radical of the formula

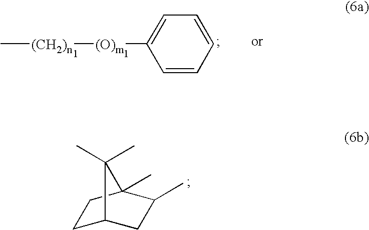

R₃₂, R₃₃ and R₃₄, independently of one another, are hydrogen; hydroxyl; C₁-C₃₀alkyl; or C₂-C₃₀alkenyl;
R₃₅ is hydrogen; or C₁-C₅alkyl;
   m₁ is 0 or 1; and
   n₁ is 1 to 5.

Preferred compounds correspond to the formula

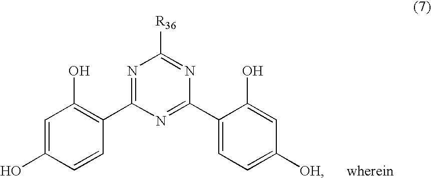

### R₃₆ is

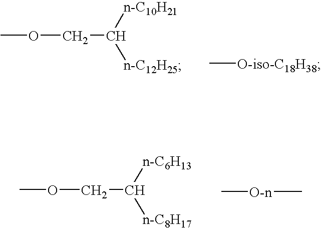

C₁₈H₃₇; or —O-2-ethylhexyl; —O—(CH₂)₃—N(C₂H₅)₂;

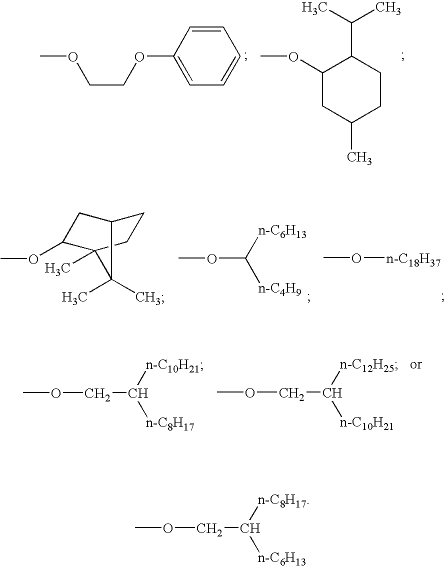

Further preferred triazine derivatives according to component (a) are those compounds having one of the formulae

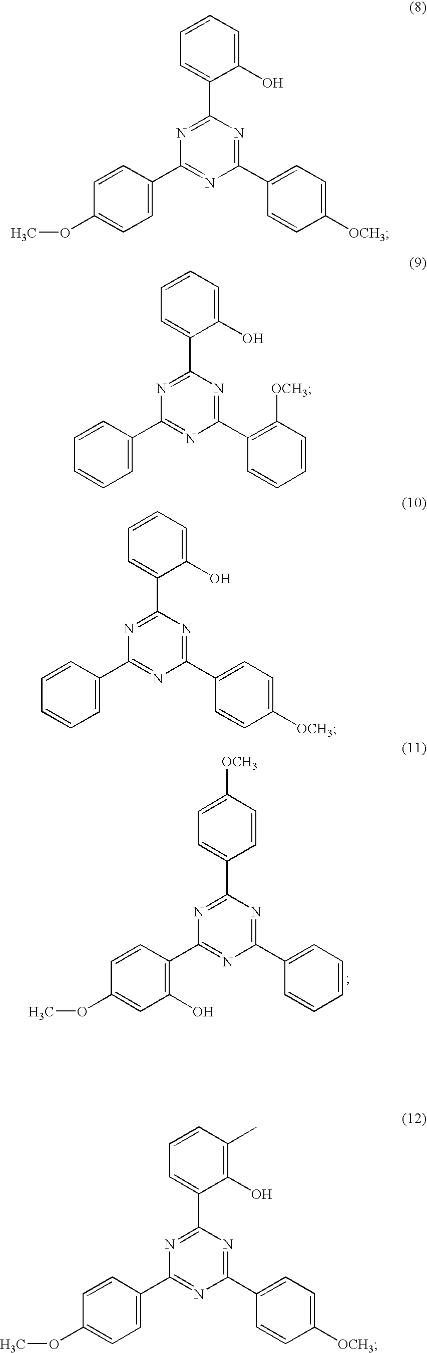

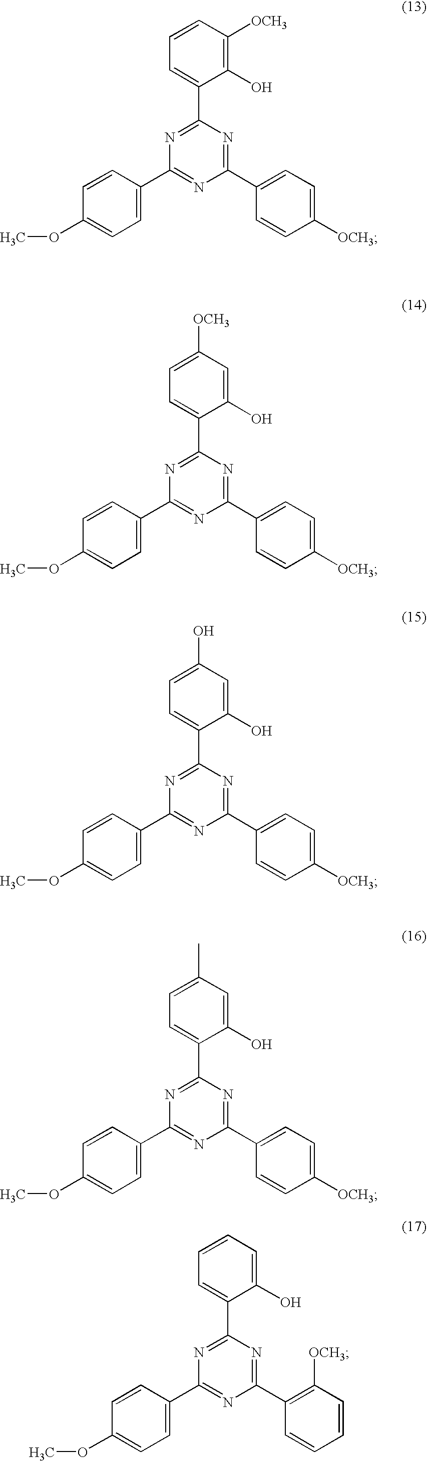

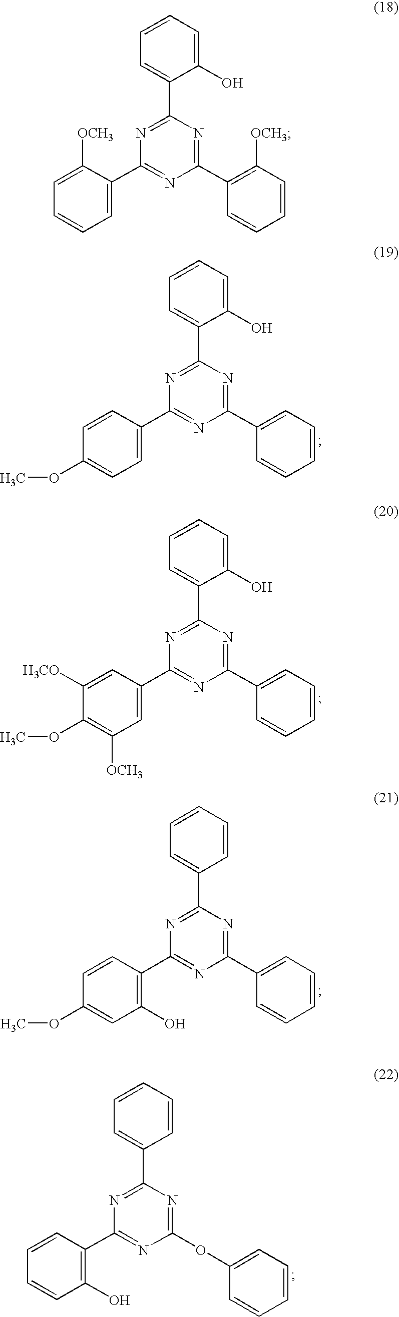

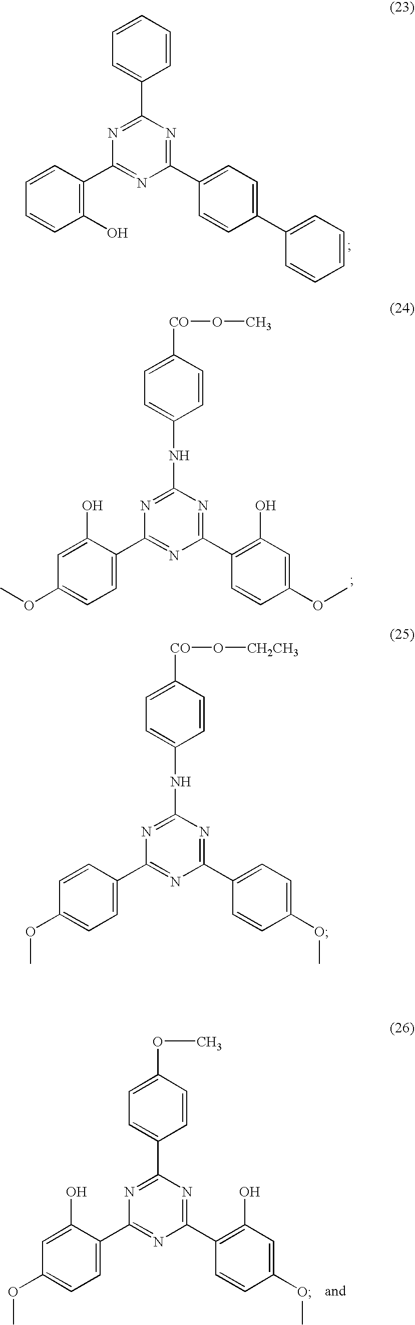

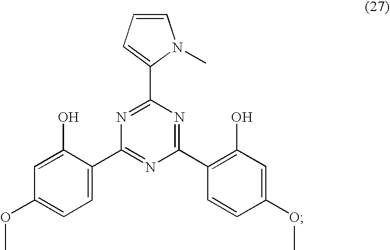

as well as 2,4,6-tris(diisobutyl-4′-aminobenzalmalonate)-s-triazine and 2,4-bis(diisobutyl-4-aminobenzalmalonate)-6-(4′-aminobenzylidenecamphor)-s-triazine.

Particularly preferred compounds of formula (1) are those having the formula:

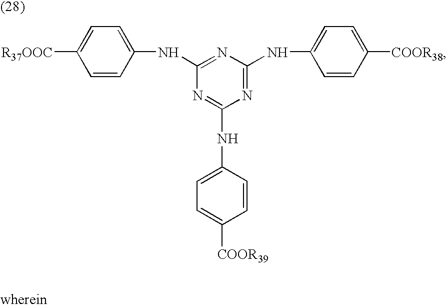

R₃₇, R₃₈ and R₃₉, independently from each other are hydrogen; an alkali metal; or an ammonium group N⁺(R₄₀)₄;
R₄₀ is hydrogen; or an organic radical; C₁-C₃alkyl; or a polyoxyethylene radical which contains from 1 to 10 ethylene oxide units and the terminal OH group of which may be etherified by a C₁-C₃alcohol.

In relation to the compounds of formula (28), when R₃₇, R₃₈ and R₃₉ is an alkali metal it is preferably potassium or, especially sodium; when R₃₇, R₃₈ and R₃₉ is a group N(R₄₀)₄ in which R₃₀ has its previous significance, it is preferably a mono-, di- or tri-C₁-C₄alkylammonium salt, a mono-, di- or tri-C₂-C₄alkanolammonium salt or a C₁-C₃alkyl ester thereof; when R₄₀ is a C₁-C₃alkyl group, it is preferably a C₁-C₂alkyl group, more preferably a methyl group; and when R₃₀ is polyoxyethylene group, this preferably contains from 2-6 ethylene oxide units.

One preferred class of benzotriazole micronized organic UV absorbers is that having the formula

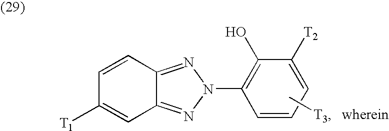

T₁ is C₁-C₃alkyl or, preferably, hydrogen; or a radical of formula

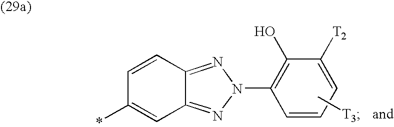

T₂ and T₃, independently from each other are C₁-C₁₂alkyl, preferably i-octyl; or C₁-C₄alkyl substituted by phenyl, preferably α,α-dimethylbenzyl.

A further preferred class of benzotriazole micronized organic UV absorbers corresponds to the formula

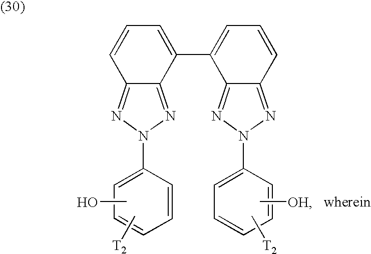

T₂ has its previous significance.

A still further preferred class of benzotriazole micronized organic UV absorbers corresponds to the formula

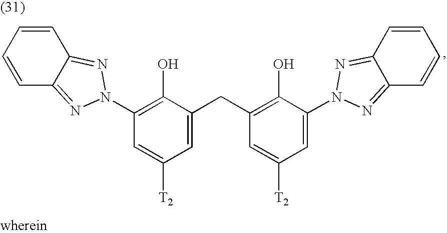

T₂ is hydrogen; C₁-C₁₂alkyl, preferably iso-octyl, or C₁-C₄alkyl substituted by phenyl, preferably α,α-dimethylbenzyl.

A preferred class of vinyl group-containing amide micronized organic UV absorbers corresponds to the formula:
(32) R₄₁-(Y)ₘ—CO—C(R₄₂)═C(R₄₃)—N(R₄₄)(R₄₅), wherein
R₄₁ is C₁-C₃alkyl, preferably C₁-C₂alkyl, or phenyl optionally substituted by one, two or three substituents selected from OH, C₁-C₃alkyl, C₁-C₃alkoxy or CO—OR₄₆,
R₄₆ C₁-C₃alkyl;
R₄₂, R₄₃, R₄₄ and R₄₅ are the same or different and each is C₁-C₃alkyl, preferably C₁-C₂alkyl; or hydrogen;

### Y is —NH—; or —O—; and

m is 0; or 1.

Preferred compounds of formula (32) are 4-methyl-3-penten-2-one, ethyl-3-methylamino-2-butenoate, 3-methylamino-1-phenyl-2-buten-1-one and 3-methylamino-1-phenyl-2-buten-1-one.

A preferred class of cinnamic acid amide micronized organic UV absorbers corresponds to the formula:

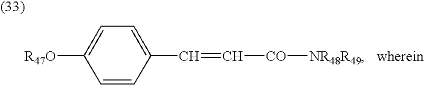

R₄₇ is hydroxy or C₁-C₄alkoxy, preferably methoxy or ethoxy;
R₄₈ is hydrogen or C₁-C₄alkyl, preferably methyl or ethyl; and
R₄₉ is —(CONH)ₘ-phenyl in which m is 0 or 1 and the phenyl group is optionally substituted by one, two or three substituents selected from OH, C₁-C₃alkyl, C₁-C₃alkoxy or CO—OR₅₀; and
R₅₀ is C₁-C₄alkyl.

A preferred class of sulfonated benzimidazole micronized organic UV absorbers corresponds to the formula

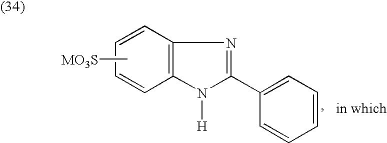

M is hydrogen; or an alkali metal, preferably sodium, an alkaline earth metal, such as magnesium or calcium, or zinc.

Further preferred classes of micronized or micronizable UV absorbers used for the present invention are:
p-aminobenzoic acid derivatives, typically 2-ethylhexyl-4-dimethylaminobenzoate
salicylic acid derivatives, typically 2-ethylhexyl salicylate; homosalates; and isopropyl sylicylates;
benzophenone derivatives, typically 2-hydroxy-4-methoxybenzophenone;
dibenzoylmethane derivatives, typically 1-(4-tert-butylphenyl)-3-(4-methoxyphenyl)-propane-1,3-dione;
diphenylacrylates, typically 2-ethylhexyl-2-cyano-3,3-diphenylacrylate and 3-(benzo-furanyl)-2-cyanoacrylate;
3-imidazol-4-yl-acrylic acid and 3-imidazol-4-yl-acrylate;
benzofurane derivatives, preferably 2-(p-aminophenyl)benzofuran derivatives, disclosed in EP-A-582 189, U.S. Pat. No. 5,338,539, U.S. Pat. No. 5,518,713 and EP-A-613 893;
polymeric UV absorbers, such as the benzylidenemalonate derivatives described, inter alia in EPA-709 080;
cinnamic acid derivatives, typically the 2-ethylhexyl-4-methoxycinnamate or isoamylate or cinnamic acid derivatives disclosed, inter alia, in U.S. Pat. No. 5,601,811 and WO 97/00851;
camphor derivatives, typically 3-(4′-methyl)benzylidenebornan-2-one, 3-benzylidene-bornan-2-one, N-[2(and 4)-2-oxyborn-3-ylidenemethyl)benzyl]acrylamide polymer, 3-(4′-trimethylammonium)benzylidenebornan-2-one methylsulfate, 3,3′-(1,4-phenylenedimethine)-bis(7,7-dimethyl-2-oxobicyclo-[2.2.1]heptane-1-methanesulfonic acid) and the salts thereof, 3-(4′-sulfo)benzylidenebornan-2-one and the salts thereof;
2-phenylbenzimidazole-5-sulfonic acids and the salts thereof; and
menthyl-o-aminobenzoate.

The micronized organic UV absorber, component (a), is preferably produced by the method described in GB-A-2303549, namely by a process which comprises grinding the corresponding organic UV absorber, in coarse particle form, in a grinding apparatus, in the presence of 1 to 50%, preferably 5 to 40% by weight, based on the micronized organic UV absorber, of an alkyl polyglucoside having the formula CₙH₂ₙ₊₁(C₆H₁₀O₅)ₓH, in which n is an integer ranging from 8 to 16 and x is the mean polymerization level of the glucoside moiety (C₆H₁₀O₅) and ranges from 1.4 to 1.6, or an ester thereof.

Any known process suitable for the preparation of microparticles can be used for the preparation of the micronised UV absorbers, for example wet-milling, wet-kneading, spray-drying from a suitable solvent, by the expansion according to the RESS process (Rapid Expansion of Supercritical Solutions), by reprecipitation from suitable solvents, including supercritical fluids (GASR process=Gas Anti-Solvent Recrystallization/PCA process=Precipitation with Compressed Anti-solvents).

The micronized UV absorbers of component (a) so obtained usually have an average particle size from 0.02 to 2, preferably from 0.03 to 1.5, and more especially from 0.05 to 1.0 micrometer.

The micronizable UV absorbers according to component (a) can also be used as dry substrates in powder form.

The sunscreen composition according to the present invention may additionally contain one or more than one further non-micronized UV filter or UV absorbers as listed in Tables 1 and 2.

The non-micronized UV absorbers as described in Tables 1 and 2 below may be added to the sunscreen composition according to the present invention in amounts from 0.01 to 25% based on weight. One or more of these UV absorbers can be used, inter alia, to improve the solubility or to increase UV absorption of the instant sunscreen composition.

TABLE 1
Suitable non-micronized UV filter substances which can be used according to
the present invention

p-aminobenzoic acid derivatives, for example 4-dimethylaminobenzoic acid 2-
ethylhexyl ester;
salicylic acid derivatives, for example salicylic acid 2-ethylhexyl ester;
benzophenone derivatives, for example 2-hydroxy-4-methoxybenzophenone and its
5-sulfonic acid derivative;
dibenzoylmethane derivatives, for example 1-(4-tert-butylphenyl)-3-(4-methoxyphenyl)-
propane-1,3-dione;
diphenylacrylates, for example 2-ethylhexyl 2-cyano-3,3-diphenylacrylate, and 3-
(benzofuranyl) 2-cyanoacrylate;
3-imidazol-4-ylacrylic acid and esters;
benzofuran derivatives, especially 2-(p-aminophenyl)benzofuran derivatives, described
in EP-A-582 189, US-A-5 338 539, US-A-5 518 713 and EP-A-613 893;
polymeric UV absorbers, for example the benzylidene malonate derivatives described
in EP-A-709 080;
cinnamic acid derivatives, for example the 4-methoxycinnamic acid 2-ethylhexyl ester
and isoamyl ester or cinnamic acid derivatives described in US-A-5 601 811 and
WO 97/00851;
camphor derivatives, for example 3-(4′-methyl)benzylidene-bornan-2-one, 3-benzyl-
idenebornan-2-one, N-[2(and 4)-2-oxyborn-3-ylidene-methyl)-benzyl]acrylamide
polymer, 3-(4′-trimethylammonium)-benzylidene-bornan-2-one methyl sulfate, 3,3′-(1,4-
phenylenedimethine)-bis(7,7-dimethyl-2-oxo-bicyclo[2.2.1]heptane-1-methanesulfonic
acid) and salts, 3-(4′-sulfo)benzylidene-bornan-2-one and salts; camphorbenzalkonium
methosulfate;
hydroxyphenyltriazine compounds, for example 2-(4′-methoxyphenyl)-4,6-bis(2′-
hydroxy-4′-n-octyloxyphenyl)-1,3,5-triazine; 2,4-bis{[4-(3-(2-propyloxy)-2-hydroxy-
propyloxy)-2-hydroxy]-phenyl}-6-(4-methoxyphenyl)-1,3,5-triazine; 2,4-bis{[4-(2-ethyl-
hexyloxy)-2-hydroxy]-phenyl}-6-[4-(2-methoxyethyl-carboxyl)-phenylamino]-1,3,5-
triazine; 2,4-bis{[4-(tris(trimethylsilyloxy-silylpropyloxy)-2-hydroxy]-phenyl}-6-(4-
methoxyphenyl)-1,3,5-triazine; 2,4-bis{[4-(2″-methylpropenyloxy)-2-hydroxy]-phenyl}-6-
(4-methoxyphenyl)-1,3,5-triazine; 2,4-bis{[4-(1′,1′,1′,3′,5′,5′,5′-heptamethyltrisilyl-2″-
methyl-propyloxy)-2-hydroxy]-phenyl}-6-(4-methoxyphenyl)-1,3,5-triazine; 2,4-bis{[4-(3-
(2-propyloxy)-2-hydroxy-propyloxy)-2-hydroxy]-phenyl}-6-[4-ethylcarboxy)-
phenylamino]-1,3,5-triazine;
benzotriazole compounds, for example 2,2′-methylene-bis(6-(2H-benzotriazol-2-yl)-4-
(1,1,3,3-tetramethylbutyl)-phenol;
trianilino-s-triazine derivatives, for example 2,4,6-trianiline-(p-carbo-2′-ethyl-1′-oxy)-
1,3,5-triazine and the UV absorbers disclosed in US-A-5 332 568, EP-A-517 104,
EP-A-507 691, WO 93/17002 and EP-A-570 838;
2-phenylbenzimidazole-5-sulfonic acid and salts thereof;
menthyl o-aminobenzoates;
physical sunscreens coated or not as titanium dioxide, zinc oxide, iron oxides, mica,
MnO, Fe₂O₃, Ce₂O₃, Al₂O₃, ZrO₂. (surface coatings: polymethylmethacrylate, methicone
(methylhydrogenpolysiloxane as described in CAS 9004-73-3), dimethicone, isopropyl
titanium triisostearate (as described in CAS 61417-49-0), metal soaps as magnesium
stearate (as described in CAS 4086-70-8), perfluoroalcohol phosphate as C9-15
fluoroalcohol phosphate (as described in CAS 74499-44-8; JP 5-86984, JP 4-
330007)). The primary particle size is an average of 15 nm-35 nm and the particle size
in dispersion is in the range of 100 nm-300 nm.
aminohydroxy-benzophenone derivatives disclosed in DE 10011317, EP 1133980 and
EP 1046391
phenyl-benzimidazole derivatives as disclosed in EP 1167358
the UV absorbers described in “Sunscreens”, Eds. N. J. Lowe, N. A. Shaath, Marcel
Dekker, Inc., New York and Basle or in Cosmetics & Toiletries (107), 50ff (1992) also
can be used as additional UV protective substances.

TABLE 2
Suitable specific non-micronized UV filter substances and adjuvants which can
be used according to the present invention

| No. | Chemical Name | CAS No. |
|---|---|---|
| 1 | (+/−)-1,7,7-trimethyl-3-[(4-methylphenyl)methylene]bicyclo- | 36861-47-9 |
| | [2.2.1]heptan-2-one; p-methyl benzylidene camphor | |
| 2 | 1,7,7-trimethyl-3-(phenylmethylene)bicyclo[2.2.1]heptan-2-one; | 15087-24-8 |
| | benzylidene camphor | |
| 3 | (2-Hydroxy-4-methoxyphenyl)(4-methylphenyl)methanone | 1641-17-4 |
| 4 | 2,4-dihydroxybenzophenone | 131-56-6 |
| 5 | 2,2′,4,4′-tetrahydroxybenzophenone | 131-55-5 |
| 6 | 2-Hydroxy-4-methoxy benzophenone; | 131-57-7 |
| 7 | 2-Hydroxy-4-methoxy benzophenone-5-sulfonic acid | 4065-45-6 |
| 8 | 2,2′-dihydroxy-4,4′-dimethoxybenzophenone | 131-54-4 |
| 9 | 2,2′-Dihydroxy-4-methoxybenzophenone | 131-53-3 |
| 10 | Alpha-(2-oxoborn-3-ylidene)toluene-4-sulphonic acid and its | 56039-58-8 |
| | salts; Mexoryl SL | |
| 11 | 1-[4-(1,1-dimethylethyl)phenyl]-3-(4-methoxyphenyl)propane-1,3- | 70356-09-1 |
| | dione; avobenzone | |
| 12 | Methyl N,N,N-trimethyl-4-[(4,7,7-trimethyl-3- | 52793-97-2 |
| | oxobicyclo[2,2,1]hept-2-ylidene)methyl]anilinium sulphate; | |
| | Mexoryl SO | |
| 22 | 3,3,5-Trimethyl cyclohexyl-2-hydroxy benzoate; homosalate | 118-56-9 |
| 23 | Isopentyl p-methoxycinnamate; isoamyl methoxy cinnamate | 71617-10-2 |
| 27 | Menthyl-o-aminobenzoate | 134-09-8 |
| 28 | Menthyl salicylate | 89-46-3 |
| 29 | 2-Ethylhexyl 2-cyano,3,3-diphenylacrylate; octocrylene | 6197-30-4 |
| 30 | 2-ethylhexyl 4-(dimethylamino)benzoate | 21245-02-3 |
| 31 | 2-ethylhexyl 4-methoxycinnamate; octyl methoxy cinnamte | 5466-77-3 |
| 32 | 2-ethylhexyl salicylate | 118-60-5 |
| 33 | Benzoic acid, 4, 4′, 4″-(1,3,5-triazine-2,4,6-triyltrimino)tris-, | 88122-99-0 |
| | tris(2-ethylhexyl)ester; 2,4,6-Trianilino-(p-carbo-2′-ethylhexyl-1′- | |
| | oxi)-1,3,5-triazine; octyl triazone | |
| 34 | 4-aminobenzoic acid | 150-13-0 |
| 35 | Benzoic acid, 4-amino-, ethyl ester, polymer with oxirane | 113010-52-9 |
| 38 | 2-phenyl-1H-benzimidazole-5-sulphonic acid; | 27503-81-7 |
| | phenylbenzimidazolsulfonic acid | |
| 39 | 2-Propenamide, N-[[4-[(4,7,7-trimethyl-3-oxobicyclo[2.2.1]hept-2- | 147897-12-9 |
| | ylidene)methyl]phenyl]methyl]-, homopolymer | |
| 40 | Triethanolamine salicylate | 2174-16-5 |
| 41 | 3,3′-(1,4-phenylenedimethylene)bis[7,7-dimethyl-2-oxo- | 90457-82-2 |
| | bicyclo[2.2.1]heptane-1 methanesulfonic acid]; Cibafast H | |
| 42 | Titanium dioxide | 13463-67-7 |
| 44 | Zinc oxide | 1314-13-2 |
| 45 | 2,4-bis{[4-(2-ethylhexyloxy)-2-hydroxy]-phenyl}-6-(4- | 187393-00-6 |
| | methoxyphenyl)-(1,3,5)-triazine; Tinosorb S | |
| 46 | 1H-Benzimidazole-4,6-disulfonic acid, 2,2′-(1,4-phenylene)bis-, | 180898-37-7 |
| | disodium salt | |
| 47 | Benzoic acid, 4,4′-[[6-[[4-[[(1,1- | 154702-15-5 |
| | dimethylethyl)amino]carbonyl]phenyl]amino]1,3,5-triazine-2,4- | |
| | diyl]diimino]-, bis(2-ethylhexyl)ester; diethylhexyl butamido | |
| | triazone; Uvasorb HEB | |
| 48 | Phenol, 2-(2H-benzotriazol-2-yl)-4-methyl-6-[2-methyl-3-[1,3,3,3- | 155633-54-8 |
| | tetramethyl-1-[(trimethylsilyl)oxy]disiloxanyl]propyl]-; drometrizole | |
| | trisiloxane; Mexoryl XL | |
| 49 | Dimethicodiethylbenzalmalonate; Polysilicone 15; Parsol SLX | 207574-74-1 |
| 50 | Benzenesulfonic acid, 3-(2H-benzotriazol-2-yl)-4-hydroxy-5-(1- | 92484-48-5 |
| | methylpropyl)-, monosodium salt; Tinogard HS | |
| 51 | Benzoic acid, 2-[4-(diethylamino)-2-hydroxybenzoyl]-, hexyl ester; | 302776-68-7 |
| | Uvinul a plus | |
| 52 | 1-Dodecanaminium, N-[3-[[4-(dimethylamino)benzoyl]amino]- | 156679-41-3 |
| | propyl]N,N-dimethyl-, salt with 4-methylbenzenesulfonic acid | |
| | (1:1); Escalol HP610 | |
| 53 | 1-Propanaminium, N,N,N-trimethyl-3-[(1-oxo-3-phenyl-2- | 177190-98-6 |
| | propenyl)amino]-, chloride | |
| 54 | 1H-Benzimidazole-4,6-disulfonic acid, 2,2′-(1,4-phenylene)bis- | 170864-82-1 |
| 55 | 1,3,5-Triazine, 2,4,6-tris(4-methoxyphenyl)- | 7753-12-0 |
| 56 | 1,3,5-Triazine, 2,4,6-tris[4-[(2-ethylhexyl)oxy]phenyl]- | 208114-14-1 |
| 57 | 1-Propanaminium, 3-[[3-[3-(2H-benzotriazol-2-yl)-5-(1,1- | 340964-15-0 |
| | dimethylethyl)-4-hydroxyphenyl]-1-oxopropyl]amino]-N,N-diethyl- | |
| | N-methyl-, methyl sulfate (salt) | |
| 58 | 2-Propenoic acid, 3-(1H-imidazol-4-yl)- | 104-98-3 |
| 59 | Benzoic acid, 2-hydroxy-, [4-(1-methylethyl)phenyl]methyl ester | 94134-93-7 |
| 60 | 1,2,3-Propanetriol, 1-(4-aminobenzoate); glyceryl PABA | 136-44-7 |
| 61 | Benzeneacetic acid, 3,4-dimethoxy-a-oxo- | 4732-70-1 |
| 62 | 2-Propenoic acid, 2-cyano-3,3-diphenyl-, ethyl ester | 5232-99-5 |
| 63 | Anthralinic acid, p-menth-3-yl ester | 134-09-8 |
| 64 | 2,2′-bis(1,4-phenylene)-1H-benzimidazole-4,6-disulphonic acid | 349580-12-7, |
| | mono sodium salt or Disodium phenyl dibenzimidazole | |
| | tetrasulfonate or Neoheliopan AP | |
| 65 | 1,3,5-Triazine-2,4,6-triamine, N,N′-bis[4-[5-(1,1-dimethylpropyl)- | 288254-16-0 |
| | 2-benzoxazolyl]phenyl]-N″-(2-ethylhexyl)- or Uvasorb K2A | |
| 66 | Merocyanine derivatives as described in WO 2004006878 and in | |
| | IPCOM000022279D | |
| 67 | 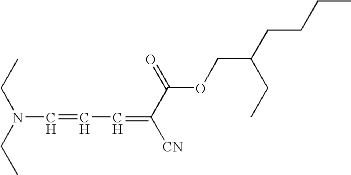 | |
| 68 | sterols (cholesterol, lanosterol, phytosterols), as described in | |
| | WO0341675 | |
| 69 | mycosporines and/or mycosporine-like amino acids as described | |
| | in WO2002039974, e.g. Helioguard 365 from Milbelle AG, | |
| | isolated mycosporine like amino acids from the red alga porphyra | |
| | umbilicalis (INCI: Porphyra Umbilicalis) that are encapsulated into | |
| | liposomes,) | |
| 70 | alpha-lipoc-acid as described in DE 10229995 | |
| 71 | synthetic organic polymers as described in EP 1371358, [0033]- | |
| | [0041] | |
| 72 | phyllosilicates as described in EP 1371357 [0034]-[0037] | |
| 73 | silica compounds as described in EP1371356, [0033]-[0041] | |
| 74 | inorganic particles as described in DE10138496 [0043]-[0055] | |
| 75 | latex particles as described in DE10138496 [0027]-[0040] | |
| 76 | 1H-Benzimidazole-4,6-disulfonic acid, 2,2′-(1,4-phenylene)bis-, | 180898-37-7 |
| | disodium salt; Bisimidazylate; Neo Heliopan APC | |

The UV screening agent of component (a) is present in the sunscreen composition in amounts from about 0.01 weight % to about 50 weight % based on the weight of the total composition. Additionally, the UV screening agent of component (a) is present in the sunscreen composition in amounts from about 0.1 weight % to about 30 weight % based on the weight of the total composition. Typically, UV screening agent of component (a) is present in the sunscreen composition in amounts from about 1 weight % to about 20 weight % based on the weight of the total composition. Typically, UV screening agent of component (a) is present in the sunscreen composition in amounts from about 1 weight % to about 5 weight % based on the weight of the total composition.

Typically, sunscreen formulations contain compositions of several UVA, UVB or broad-spectrum sunscreen actives: organic that are oil or water soluble, inorganic or organic particulates.

The term “effective amount” means for example the amount necessary to achieve the desired effect.

The select copolymers of component (b) formula (I) according to the instant invention maybe derived from at least two different monomers. Another aspect of the instant invention is the select copolymers of component (b) may be derived from at least three different monomers. Another aspect of the instant invention is the select copolymers of component (b) may be derived from at least four different monomers.

Additionally, the select copolymers of component (b) formula (I) according to the instant invention maybe derived from one monomer. For example, A is a homopolymer and the group “—O-G” is grafted or reacted onto A, thus producing the select copolymer of component b) formula (I).

The select copolymers of component (b) formula (I) can be used in conjunction with other polymers or copolymers in a sunscreen formulation or personal care composition; for example, the polymers listed in U.S. Pat. No. 6,409,998 and/or in US 2006/0104923.

The polymer A of formula (I) may be derived from monomers that are selected from the group consisting of anionic water soluble monomers, nonionic water soluble monomers, cationic water soluble monomers and water insoluble monomers.

For example, the anionic water-soluble monomers of polymer A of formula (I) may be chosen from monomers of carboxylic acids comprising ethylenic unsaturation, such as acrylic acid, methacrylic acid, itaconic acid, fumaric acid, crotonic acid and maleic acid, 2-acrylamido-2-methylpropanesulfonic acid, styrenesulfonic acid, vinylsulfonic acid and vinylphosphonic acid.

The nonionic water-soluble monomers of polymer A of formula (I) may, for example, be chosen from acrylamides, N—(C1-6 alkylated)acrylamides and N,N-di(C1-3 alkylated)acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising at least one cyclic group chosen from cyclic groups comprising from 4 to 9 carbon atoms, vinyl alcohol (copolymerized in the form of vinyl acetate and then hydrolyzed), ethylene oxide, hydroxyethyl acrylate, hydroxypropyl acrylate, hydroxyethyl methacrylate and hydroxypropyl methacrylate.

The cationic water-soluble monomers of polymer A of formula (I) may, for example, be chosen from dimethyldiallylammonium chloride, methylvinylimidazolium chloride, 2-vinylpyridine, 4-vinylpyridine, vinylimidazole, 2-methyl-N-vinylimidazole, vinylpyrrolidone, vinylcarbazole, 2-methyl-5-vinylpyridine, N—(C₁₋₄ alkyl)-4-vinylpyridinium halides, such as N-methyl-4-vinylpyridinium iodide, vinylamine and monomers of the following formula: H2C═CR1—CO—X2 wherein: R1 is chosen from a hydrogen atom and a methyl group; X2 is chosen from linear and branched C₁₋₆ hydrocarbonaceous groups carrying at least one entity chosen from primary, secondary and tertiary amine functional groups; quaternary nitrogen atoms; groups of formula NHR2; and groups of formula NR2R3, wherein R2 and R3, which may be identical or different, can each be chosen from linear and branched C1-6 hydrocarbonaceous groups carrying at least one entity chosen from primary, secondary and tertiary amine functional groups and quaternary nitrogen atoms.

The cationic water-soluble monomers of polymer A of formula (I) may, for example, be chosen from 1-(2-hydroxyethyl)-pyrrolidine, 2-(1-pyrrolidyl)-ethylamine, 2-(1-piperidyl)-ethylamine, 1-(2-hydroxyethyl)-piperidine, 1-(2-aminopropyl)-piperidine, N-(2-hydroxyethyl)-hexamethylenimine, 4-(2-hydroxyethyl)-morpholine, 2-(4-morpholinyl)-ethylamine, 4-(3-aminopropyl)-morpholine, 1-(2-hydroxyethyl)-piperazine, 1-(2-aminoethyl)-piperazine, 1-(2-hydroxyethyl)-2-alkylimidazoline, 1-(3-aminopropyl)-imidazole, (2-aminoethyl)-pyridine, (2-hydroxyethyl)-pyridine, (3-hydroxypropyl)-pyridine, (hydroxymethyl)-pyridine, N-methyl-2-hydroxy-methyl-piperidine, 1-(2-hydroxyethyl)-imidazole, 2-amino-6-methoxybenzothiazole, 4-aminomethyl-pyridine, 4-amino-2-methoxypyrimidine, 2-mercaptopyrimidine, 2-mercapto-benzimidazole, 3-amino-1,2,4-triazole, 2-isopropyl-imidazole, 2-ethyl-imidazole, 4-methyl-imidazole, 2-methyl-imidazole, 2-ethyl-4-methyl-imidazole, 2-phenyl-imidazole and 4-nitro-imidazole.

The water-insoluble monomers of polymer A of formula (I) may, for example, be chosen from vinylaromatic monomers, such as styrene and its alkylated derivatives, for example, 4-butylstyrene, .alpha.-methylstyrene and vinyltoluene; dienes, such as butadiene and 1,3-hexadiene; alkylated derivatives of dienes, such as isoprene and dimethylbutadiene; chloroprene; C1-10 alkyl, C6-10 aryl and C7-20 aralkyl acrylates; C1-10 alkyl, C6-10 aryl and C7-20 aralkyl methacrylates, for example, methyl, ethyl, n-butyl, 2-ethylhexyl, tert-butyl, isobornyl, phenyl and benzyl(meth)acrylates; vinyl acetate; vinyl ethers of formula CH2=CH—O—R and allyl ethers of formula CH2=CH—CH2—O—R wherein R is chosen from C1-6 alkyl groups; acrylonitrile; vinyl chloride; vinylidene chloride; caprolactone; ethylene, propylene, and fluorinated vinyl monomers; and vinyl monomers comprising at least one perfluorinated chain, such as fluoroalkyl acrylates and methacrylates and alkyl .alpha.-fluoroacrylates.

The water-insoluble monomers of polymer A of formula (I) are selected from the group consisting of styrene, methylacrylate, ethylacrylate, butylacrylate, isobutylacrylate, tert. butylacrylate, hydroxyethylacrylate, hydroxypropylacrylate, dimethylaminoethylacrylate, glycidylacrylates, methyl(meth)acrylate, ethyl(meth)acrylate, butyl(meth)acrylate, hydroxyethyl(meth)acrylate, hydroxypropyl(meth)acrylate, dimethylaminoethyl(meth)acrylate, glycidyl(meth)acrylates, acrylonitrile, acrylamide, methacrylamide, dimethylaminopropyl-methacrylamide, cyclohexyl methacrylate, isobornyl methacrylate, 2-ethyl hexyl acrylate, cetyl methacrylate, stearyl methacrylate, behenyl methacrylate, polypropylene glycol monomethacrylate, polyethylene glycol monomethacrylate and EO-PO-monomethacrylate.

The select copolymers of component b) formula (I) according to the instant invention maybe be crosslinked by multifunctional monomers. These multifunctional monomers are selected from the group consisting of divinyl benzene, trivinylbenzene, divinyltoluene, divinylpyridine, divinylnaphthalene divinylxylene, ethyleneglycol di(meth)acrylate, trimethylolpropane tri(meth)acrylate, diethyleneglycol divinyl ether, trivinylcyclohexane, allyl(meth)acrylate, diethyleneglycol di(meth)acrylate, propyleneglycol di(meth)acrylate, 2,2-dimethylpropane-1,3-di(meth)acrylate, 1,3-butylene glycol di(meth)acrylate, 1,4-butanediol di(meth)acrylate, 1,6-hexanediol di(meth)acrylate, tripropylene glycol di(meth)acrylate, triethylene glycol di(meth)acrylate, tetraethylene glycol di(meth)acrylate, polyethylene glycol di(meth)acrylates, polyethylene glycol 200 di(meth)acrylate, polyethylene glycol 600 di(meth)acrylate, ethoxylated bisphenol A di(meth)acrylate, poly(butanediol) di(meth)acrylate, pentaerythritol tri(meth)acrylate, trimethylolpropane triethoxy tri(meth)acrylate, glyceryl propoxy tri(meth)acrylate, pentaerythritol tetra(meth)acrylate, dipentaerythritol monohydroxypenta(meth)acrylate, divinyl silane, trivinyl silane, dimethyl divinyl silane, divinyl methyl silane, methyl trivinyl silane, diphenyl divinyl silane, divinyl phenyl silane, trivinyl phenyl silane, divinyl methyl phenyl silane, tetravinyl silane, dimethyl vinyl disiloxane, poly(methyl vinyl siloxane), poly(vinyl hydro siloxane), poly(phenyl vinyl siloxane), and mixtures thereof. The amount of crosslinking monomer(s) may be from about 0 weight percent to about 20 weight percent based on the total weight of the copolymer. The amount of crosslinking monomer(s) may be from about 0.01 weight percent to about 10 weight percent based on the total weight of the copolymer. The amount of crosslinking monomer(s) may be from about 0.01 weight percent to about 5 weight percent based on the total weight of the copolymer.

Another embodiment of the instant invention is a copolymer of formula (I) wherein z is from about 0.001% to about 80% by weight of the copolymer and x is from about 20% to about 99.999% by weight of the copolymer. Another embodiment of the instant invention is a copolymer of formula (I) wherein z is from about 0.001% to about 40% by weight of the copolymer and x is from about 60% to about 99.999% by weight of the copolymer. Another embodiment of the instant invention is a copolymer of formula (I) wherein z is from about 0.001% to about 20% by weight of the copolymer and x is from about 80% to about 99.999% by weight of the copolymer. Another embodiment of the instant invention is a copolymer of formula (I) wherein z is from about 0.001% to about 10% by weight of the copolymer and x is from about 90% to about 99.999% by weight of the copolymer. Another embodiment of the instant invention is a copolymer of formula (I) wherein z is from about 0.001% to about 5% by weight of the copolymer and x is from about 95% to about 99.999% by weight of the copolymer.

The weight-average molecular weight of the select copolymer of component (b) formula (I) exhibits a weight-average molecular weight of about 500 Daltons to about 1,000,000 Daltons. In another aspect of the instant invention, the weight-average molecular weight of the select copolymer of component (b) formula (I) exhibits a weight-average molecular weight of about 500 Daltons to about 500,000 Daltons. In yet another aspect of the instant invention, the weight-average molecular weight of the select copolymer of component (b) formula (I) exhibits a weight-average molecular weight of about 500 Daltons to about 100,000 Daltons. In still another aspect of the instant invention, the weight-average molecular weight of the select copolymer of component (b) formula (I) exhibits a weight-average molecular weight of about 1000 Daltons to about 75,000 Daltons.

The select copolymer of component (b) formula (I) is present in the sunscreen composition in amounts from about 0.01 weight % to about 50 weight % based on the weight of the total composition. In another aspect of the instant invention, the select copolymer of component (b) formula (I) is present in the sunscreen composition in amounts from about 0.1 weight % to about 25 weight % based on the weight of the total composition. In still another aspect of the instant invention, the select copolymer of component (b) formula (I) is present in the sunscreen composition in amounts from about 0.1 weight % to about 10 weight % based on the weight of the total composition.

Another embodiment of the instant invention are select copolymers of component (b) formula (I) that contain less than 250 ppm of residual monomers. Another embodiment of the instant invention are select copolymers of component (b) formula (I) that contain less than 200 ppm of residual monomers. Another embodiment of the instant invention are select copolymers of component (b) formula (I) that contain less than 100 ppm of residual monomers. Another embodiment of the instant invention are select copolymers of component (b) formula (I) that contain less than 50 ppm of residual monomers. Another embodiment of the instant invention are select copolymers of component (b) formula (I) that contain less than 5 ppm of residual monomers.

The select copolymers of the instant invention are water-dispersible and can be distributed throughout the aqueous phase or the oil phase of the instant compositions.

The select copolymers of component (b) can be prepared in the conventional manner, e.g., by mass or solution polymerization. The polymerization in a solvent is preferred in view of the controllability of the polymerization and the viscosity of the final product. Suitable solvents are DMSO, THF, DMF, ethyl, propyl, butyl, acetate, benzene, toluene, xylene, N-butanol, isobutanol, isopropanol, MEK, MIBK, acetone, etc.

The monomers are preferably polymerized using a radical reaction, by addition of peroxides, optionally in the presence of redox systems.

The polymerization time of the select copolymer of component (b) depends on the temperature and the desired final product properties but is preferably within the range of from 0.5 to 10 hours at temperatures ranging from about 50° C. to about 190° C. The polymerization can be carried out continuously, discontinuously or semicontinuously. If it is preferred to obtain a polymer chain having random distribution of monomers, all of the monomers together will be preferably added to the reaction mixture. This may be done in one portion or in the course of time.

On the basis of the reactivity of the monomers, which is known, a skilled artisan can control the polymerization so as to obtain the desired distribution.

The sunscreen compositions according to the invention may also contain agents for tanning and/or for artificial tanning of the skin (self-tanning agents), such as, for example, dihydroxyacetone (DHA).

The sunscreen compositions according to the invention may also contain agents for lightening or brightening of the skin, such as, for example, kojic acid, arbutin.

The compositions of the invention may further comprise, cosmetically acceptable ingredients and adjuvants selected, in particular, from among fatty substances, organic solvents, thickeners, demulcents, opacifiers, colorants, effect pigments, stabilizers, emollients, antifoaming agents, moisturizing agents, antioxidants, vitamins, peptides, amino acids, botanical extracts, particulates, perfumes, preservatives, polymers, fillers, sequestrants, propellants, alkalinizing or acidifying agents or any other ingredient customarily formulated into cosmetics, in particular for the production of anti-sun/sunscreen compositions.

The fatty substances may be an oil or a wax or mixtures thereof, and they also comprise fatty acids, fatty alcohols and esters of fatty acids. The oils may be selected from among animal, vegetable, mineral or synthetic oils and, in particular, from among liquid paraffin, paraffin oil, silicone oils, volatile or otherwise, isoparaffins, polyolefins, fluorinated or perfluorinated oils. Likewise, the waxes may be animal, fossil, vegetable, mineral or synthetic waxes which are also known per se.

Exemplary organic solvents include the lower alcohols and polyols.

Of course, one skilled in this art will take care to select this or these optional additional compounds and/or their quantities such that the advantageous properties, in particular the resistance to water, the stability, which are intrinsically associated with the sunscreen compositions in accordance with the invention are not, or not substantially, altered by the addition(s) envisaged.

The sunscreen compositions of the invention may be formulated according to techniques well known to this art, in particular those suited for the preparation of emulsions of the oil-in-water or water-in-oil type.

The subject sunscreen compositions may be provided, in particular, in the form of a simple or complex (O/W, W/O, O/W/O or WIO/W) emulsion such as a cream, a milk, a gel or a gel cream, of a powder, a lotion, an ointment, a solid stick and may optionally be packaged as an aerosol and provided in the form of a foam, mousse or spray.

When an emulsion is provided, the aqueous phase thereof may comprise a nonionic vesicular dispersion prepared according to known techniques (Bangham, Standish and Watkins, J. Mol. Biol., 13, 238 (1965), FR-2,315,991 and FR-2,416,008).

The sunscreen compositions according to the invention may be formulated for protecting the human epidermis or the hair against the damaging effects of ultraviolet radiation, as an anti-sun composition or as a makeup product.

When the sunscreen compositions according to the invention are formulated for protecting the human epidermis against UV rays, or as anti-sun/sunscreen compositions, same may be provided in the form of a suspension or a dispersion in solvents or fatty substances, in the form of a nonionic vesicular dispersion or, alternatively, in the form of an emulsion, preferably of the oil-in-water type, such as a cream or a milk, in the form of an ointment, a gel, a gel cream, a solid stick, a powder, a stick, an aerosol foam or a spray.

When the sunscreen compositions according to the invention are formulated for protecting the hair against UV rays, same may be provided in the form of a shampoo, a body wash, a lotion, a gel, an alcohol-based system, an emulsion, a nonionic vesicular dispersion and may constitute, for example, a rinse-off composition to be applied before or after shampooing, before or after dyeing or bleaching, before, during or after permanent-waving or hair straightening, a hair-styling or treatment lotion or gel, a lotion or gel for blow drying or hair setting, a composition for permanent waving or straightening, dyeing or bleaching the hair.

When the subject compositions are formulated as makeup products for the eyelashes, the eyebrows or the skin, such as a treatment cream for the epidermis, foundation, lipstick, eyeshadow, blusher, mascara or eyeliner, same may be provided in a solid or pasty, anhydrous or aqueous form, such as oil-in-water or water-in-oil emulsions, nonionic vesicular dispersions or alternatively suspensions.

For example, for the anti-sun formulations in accordance with the invention which have a carrier, vehicle or diluent of the oil-in-water emulsion type, the aqueous phase (comprising in particular the hydrophilic screening agents), generally constitutes from 50% to 95% by weight, preferably from 70% to 90% by weight, relative to the total weight of the formulation, the oily phase (comprising in particular the lipophilic screening agents), from 5% to 50% by weight, preferably from 10% to 30% by weight, relative to the total weight of the formulation, and the (co)emulsifier(s) from 0.5% to 20% by weight, preferably from 2% to 10% by weight, also relative to the total weight of the formulation.

As indicated above, the present invention thus features formulating the subject emulsions for the production of cosmetic compositions for protecting the skin and/or the hair against ultraviolet radiation, in particular solar radiation.

The sunscreen composition of the instant invention may further comprise a fragrance. The term “perfume” or “fragrance” as used herein refers to odoriferous materials which are able to provide a pleasing fragrance to fabrics, and encompasses conventional materials commonly used in cosmetic compositions to counteract a malodor in such compositions and/or provide a pleasing fragrance thereto. The perfumes are preferably in the liquid state at ambient temperature, although solid perfumes are also useful, particularly cyclodextrin/perfume inclusion complexes for controlled release. Included among the perfumes contemplated for use herein are materials such as aldehydes, ketones, esters and the like which are conventionally employed to impart a pleasing fragrance to liquid and solid personal care or cosmetic compositions. Naturally occurring plant and animal oils are also commonly used as components of perfumes. Accordingly, the perfumes useful for the present invention may have relatively simple compositions or may comprise complex mixtures of natural and synthetic chemical components, all of which are intended to provide a pleasant odor or fragrance when applied to fabrics. The perfumes used in personal care or cosmetic compositions are generally selected to meet the normal requirements of odor, stability, price and commercial availability. The term “fragrance” is often used herein to signify a perfume itself, rather than the aroma imparted by such perfume.

The present invention is directed to a method of increasing the sun protection factor of a sunscreen composition wherein said method comprises incorporating into said composition an effective amount of at least one select copolymer according to formula (I)

(a) at least one UV screening agent;
(b) at least one select copolymer comprising

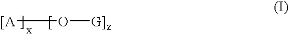

wherein
x and z represent the percentage by weight that each repeating unit or derived monomer is contained within the copolymer;
x and z add up to total 100 weight percent relative to the total weight of the copolymer;
x and z refer to repeating units;
z is from about 0.001% to about 99.999% by weight of the copolymer;
x is from about 0.001% to about 99.999% by weight of the copolymer;
A is a polymer;
G is covalently bonded to the polymer A through an oxygen linking group;
o is an oxygen atom;

### G is

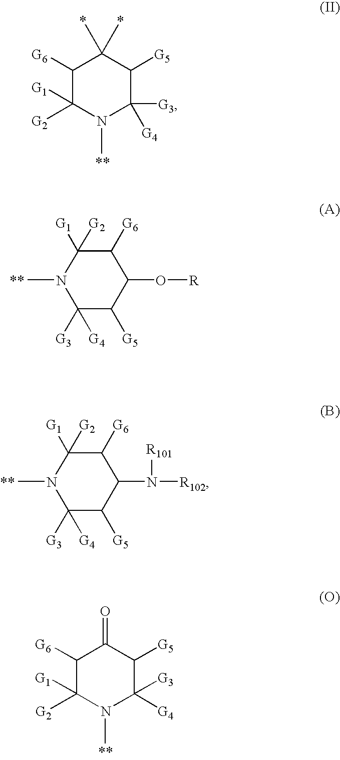

wherein
G₁, G₂, G₃, G₄ are independently C₁-C₆alkyl or G₁ and G₂ or G₃ and G₄, or G₁ and G₂ and
G₃ and G₄ together form a C₅-C₁₂cycloalkyl group;
G₅, G₆ independently are H, C₁-C₁₈alkyl, phenyl, naphthyl or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, of a cycloaliphatic carboxylic acid having 7 to 15 carbon atoms, or an alpha, beta-unsaturated carboxylic acid having 3 to 5 carbon atoms or of an aromatic carboxylic acid having 7 to 15 carbon atoms;
R₁₀₁, is C₁-C₁₂alkyl, C₅-C₇cycloalkyl, C₇-C₈aralkyl, C₂-C₁₈alkanoyl, C₃-C₅alkenoyl or benzoyl;
R₁₀₂ is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or

### G is

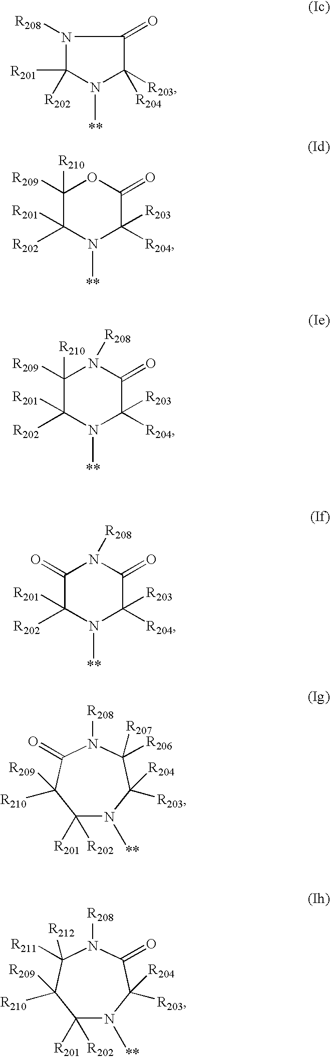

wherein R₂₀₁, R₂₀₂, R₂₀₃ and R₂₀₄ independently of each other are C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl or R₂₀₁ and R₂₀₂ and/or R₂₀₃ and R₂₀₄ together with the linking carbon atom form a C₃-C₁₂cycloalkyl radical;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by one or more OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉phenylalkyl, C₅-C₁₀heteroaryl, —C(O)—C₁-C₁₈alkyl, —O—C₁-C₁₈alkyl or —COOC₁-C₁₈alkyl;
R₂₀₉, R₂₁₀, R₂₁₁ and R₂₁₂ are independently hydrogen, phenyl or C₁-C₁₈alkyl; or

### G is

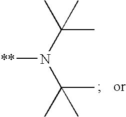

### G is

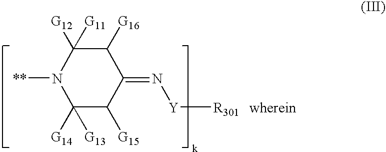

G₁₁, G₁₂, G₁₃ and G₁₄ are independently C₁-C₄alkyl or G₁₁ and G₁₂ together and G₁₃ and G₁₄ together, or G₁₁ and G₁₂ together or G₁₃ and G₁₄ together are pentamethylene;
G₁₅ and G₁₆ are each independently of the other hydrogen or C₁-C₄alkyl;
X is as defined above;
k is 1, 2, 3, or 4
Y is O or NR₃₀₂ or when k is 1 and R₃₀₁ represents alkyl or aryl Y is additionally a direct bond;
R₃₀₂ is H, C₁-C₁₈alkyl or phenyl;
if k is 1
R₃₀₁ is H, straight or branched C₁-C₁₈alkyl, C₃-C₁₈alkenyl or C₃-C₁₈alkinyl, which may be unsubstituted or substituted, by one or more OH, C₁-C₈alkoxy, carboxy, C₁-C₈alkoxycarbonyl;
C₅-C₁₂cycloalkyl or C₅-C₁₂cycloalkenyl;
phenyl, C₇-C₉phenylalkyl or naphthyl which may be unsubstituted or substituted by one or more C₁-C₈alkyl, halogen, OH, C₁-C₈alkoxy, carboxy, C₁-C₈alkoxycarbonyl; —C(O)—C₁-C₃₆alkyl, or an acyl moiety of a □,□-unsaturated carboxylic acid having 3 to 5 carbon atoms or of an aromatic carboxylic acid having 7 to 15 carbon atoms;
—SO₃⁻Q⁺, —PO(O⁻Q⁺)₂, —P(O)(OC₁-C₈alkyl₂)₂, —P(O)(OH₂)₂, —SO₂—OH, —SO₂—C₁-C₈alkyl, —CO—NH—C₁-C₈alkyl, —CONH₂, COO—C₁-C₈alkyl₂, COOH or Si(Me)₃, wherein Q⁺ is H⁺, ammonium or an alkali metal cation;
if k is 2
R₃₀₁ is C₁-C₁₈alkylene, C₃-C₁₈alkenylene or C₃-C₁₈alkinylene, which may be unsubstituted or substituted, by one or more OH, C₁-C₈alkoxy, carboxy, C₁-C₈alkoxycarbonyl; or xylylene; or
R₃₀₁ is a bisacyl radical of an aliphatic dicarboxylic acid having 2 to 36 carbon atoms, or a cycloaliphatic or aromatic dicarboxylic acid having 8-14 carbon atoms;
if k is 3,
R₃₀₁ is a trivalent radical of an aliphatic, cycloaliphatic or aromatic tricarboxylic acid; and
if k is 4, R₃₀₁ is a tetravalent radical of an aliphatic, cycloaliphatic or aromatic tetracarboxylic acid;
and, optionally
(c) other cosmetically acceptable ingredients.

The present invention is directed to a method of improved UV protection of mammalian hair and/or skin from the damaging effects of UV radiation wherein said method comprises applying to said skin and/or said hair an effective amount of a sunscreen composition comprising

(a) at least one UV screening agent;
(b) at least one select copolymer comprising

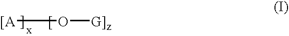

wherein
x and z represent the percentage by weight that each repeating unit or derived monomer is contained within the copolymer;
x and z add up to total 100 weight percent relative to the total weight of the copolymer;
x and z refer to repeating units;
z is from about 0.001% to about 99.999% by weight of the copolymer;
x is from about 0.001% to about 99.999% by weight of the copolymer;
A is a polymer;
G is covalently bonded to the polymer A through an oxygen linking group;
O is an oxygen atom;

### G is

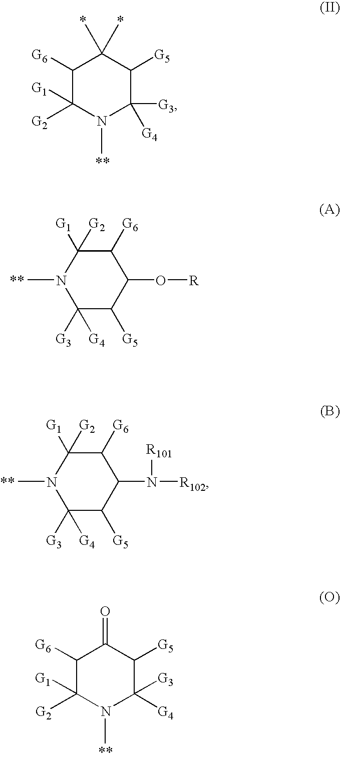

wherein
G₁, G₂, G₃, G₄ are independently C₁-C₆alkyl or G₁ and G₂ or G₃ and G₄, or G₁ and G₂ and G₃ and G₄ together form a C₅-C₁₂cycloalkyl group;
G₅, G₆ independently are H, C₁-C₁₈alkyl, phenyl, naphthyl or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, of a cycloaliphatic carboxylic acid having 7 to 15 carbon atoms, or an alpha, beta-unsaturated carboxylic acid having 3 to 5 carbon atoms or of an aromatic carboxylic acid having 7 to 15 carbon atoms;
R₁₀₁ is C₁-C₁₂alkyl, C₅-C₇cycloalkyl, C₇-C₈aralkyl, C₂-C₁₈alkanoyl, C₃-C₅alkenoyl or benzoyl;
R₁₀₂ is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or

### G is

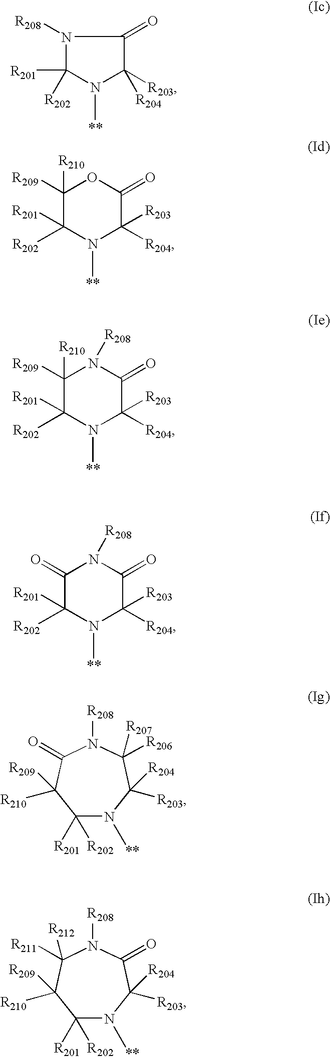

wherein R₂₀₁, R₂₀₂, R₂₀₃ and R₂₀₄ independently of each other are C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl or R₂₀, and R₂₀₂ and/or R₂₀₃ and R₂₀₄ together with the linking carbon atom form a C₃-C₁₂cycloalkyl radical;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by one or more OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉phenylalkyl, C₅-C₁₀heteroaryl, —C(O)—C₁-C₁₈alkyl, —O—C₁-C₁₈alkyl or —COOC₁-C₁₈alkyl;
R₂₀₉, R₂₁₀, R₂₁₁ and R₂₁₂ are independently hydrogen, phenyl or C₁-C₁₈alkyl; or

### G is

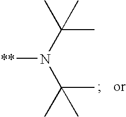

### G is

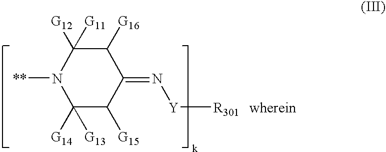

G₁₁, G₁₂, G₁₃ and G₁₄ are independently C₁-C₄alkyl or G₁₁ and G₁₂ together and G₁₃ and G₁₄ together, or G₁₁ and G₁₂ together or G₁₃ and G₁₄ together are pentamethylene;
G₁₅ and G₁₆ are each independently of the other hydrogen or C₁-C₄alkyl;
X is as defined above;
k is 1, 2, 3, or 4
Y is O or NR₃₀₂ or when k is 1 and R₃₀₁ represents alkyl or aryl Y is additionally a direct bond;
R₃₀₂ is H, C₁-C₁₈alkyl or phenyl;
if k is 1
R₃₀₁ is H, straight or branched C₁-C₁₈alkyl, C₃-C₁₈alkenyl or C₃-C₁₈alkinyl, which may be unsubstituted or substituted, by one or more OH, C₁-C₈alkoxy, carboxy, C₁-C₈alkoxycarbonyl;
C₅-C₁₂cycloalkyl or C₅-C₁₂cycloalkenyl;
phenyl, C₇-C₉phenylalkyl or naphthyl which may be unsubstituted or substituted by one or more C₁-C₈alkyl, halogen, OH, C₁-C₈alkoxy, carboxy, C₁-C₈alkoxycarbonyl;
—C(O)—C₁-C₃₆alkyl, or an acyl moiety of a □,□-unsaturated carboxylic acid having 3 to 5 carbon atoms or of an aromatic carboxylic acid having 7 to 15 carbon atoms;
—SO₃⁻Q⁺, —PO(O⁻Q⁺)₂, —P(O)(OC₁-C₈alkyl₂)₂, —P(O)(OH₂)₂, —SO₂—OH, —SO₂—C₁-C₈alkyl, —CO—NH—C₁-C₈alkyl, —CONH₂, COO—C₁-C₈alkyl₂, COOH or Si(Me)₃, wherein Q⁺ is H⁺, ammonium or an alkali metal cation;
if k is 2
R₃₀₁ is C₁-C₁₈alkylene, C₃-C₁₈alkenylene or C₃-C₁₈alkinylene, which may be unsubstituted or substituted, by one or more OH, C₁-C₈alkoxy, carboxy, C₁-C₈alkoxycarbonyl; or xylylene; or
R₃₀₁ is a bisacyl radical of an aliphatic dicarboxylic acid having 2 to 36 carbon atoms, or a cycloaliphatic or aromatic dicarboxylic acid having 8-14 carbon atoms;
if k is 3,
R₃₀₁ is a trivalent radical of an aliphatic, cycloaliphatic or aromatic tricarboxylic acid; and
if k is 4, R₃₀₁ is a tetravalent radical of an aliphatic, cycloaliphatic or aromatic tetracarboxylic acid;
and, optionally
(c) other cosmetically acceptable ingredients.

The present invention is directed to a cosmetic or dermatological composition comprising a select copolymer of formula (I)

(b) at least one select copolymer comprising

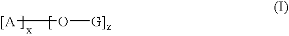

wherein
x and z represent the percentage by weight that each repeating unit or derived monomer is contained within the copolymer;
x and z add up to total 100 weight percent relative to the total weight of the copolymer;
x and z refer to repeating units;
z is from about 0.001% to about 99.999% by weight of the copolymer;
x is from about 0.001% to about 99.999% by weight of the copolymer;
A is a polymer;
G is covalently bonded to the polymer A through an oxygen linking group;
O is an oxygen atom;

### G is

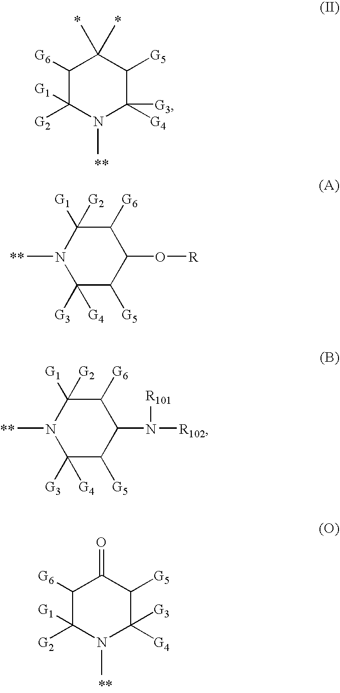

wherein
G₁, G₂, G₃, G₄ are independently C₁-C₆alkyl or G₁ and G₂ or G₃ and G₄, or G₁ and G₂ and G₃ and G₄ together form a C₅-C₁₂cycloalkyl group;
G₅, G₆ independently are H, C₁-C₁₈alkyl, phenyl, naphthyl or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, of a cycloaliphatic carboxylic acid having 7 to 15 carbon atoms, or an alpha, beta-unsaturated carboxylic acid having 3 to 5 carbon atoms or of an aromatic carboxylic acid having 7 to 15 carbon atoms;
R₁₀₁ is C₁-C₁₂alkyl, C₅-C₇cycloalkyl, C₇-C₈aralkyl, C₂-C₁₈alkanoyl, C₃-C₅alkenoyl or benzoyl;
R₁₀₂ is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or

### G is

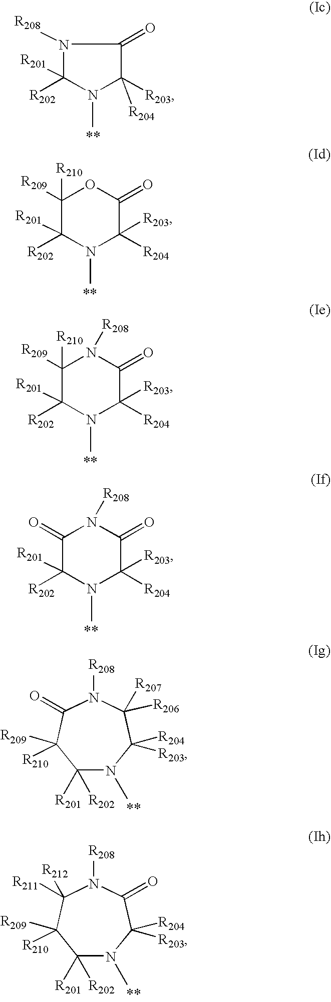

wherein R₂₀₁, R₂₀₂, R₂₀₃ and R₂₀₄ independently of each other are C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl or R₂₀, and R₂₀₂ and/or R₂₀₃ and R₂₀₄ together with the linking carbon atom form a C₃-C₁₂cycloalkyl radical;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by one or more OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉-phenylalkyl, C₅-C₁₀heteroaryl, —C(O)—C₁-C₁₈alkyl, —O—C₁-C₁₈alkyl or —COOC₁-C₁₈alkyl;
R₂₀₉, R₂₁₀, R₂₁₁ and R₂₁₂ are independently hydrogen, phenyl or C₁-C₁₈alkyl; or

### G is

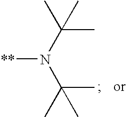

### G is

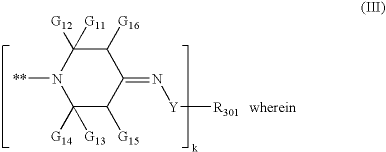

G₁₁, G₁₂, G₁₃ and G₁₄ are independently C₁-C₄alkyl or G₁₁ and G₁₂ together and G₁₃ and
G₁₄ together, or G₁₁ and G₁₂ together or G₁₃ and G₁₄ together are pentamethylene; G₁₅ and G₁₆ are each independently of the other hydrogen or C₁-C₄alkyl;
X is as defined above;
k is 1, 2, 3, or 4
Y is O or NR₃₀₂ or when k is 1 and R₃₀₁ represents alkyl or aryl Y is additionally a direct bond;
R₃₀₂ is H, C₁-C₁₈alkyl or phenyl;
if k is 1
R₃₀₁ is H, straight or branched C₁-C₁₈alkyl, C₃-C₁₈alkenyl or C₃-C₁₈alkinyl, which may be unsubstituted or substituted, by one or more OH, C₁-C₈alkoxy, carboxy, C₁-C₈alkoxycarbonyl;
C₅-C₁₂cycloalkyl or C₅-C₁₂cycloalkenyl;
phenyl, C₇-C₉phenylalkyl or naphthyl which may be unsubstituted or substituted by one or more C₁-C₈alkyl, halogen, OH, C₁-C₈alkoxy, carboxy, C₁-C₈alkoxycarbonyl;
—C(O)—C₁-C₃₆alkyl, or an acyl moiety of a □,□-unsaturated carboxylic acid having 3 to 5 carbon atoms or of an aromatic carboxylic acid having 7 to 15 carbon atoms;
—SO₃⁻Q⁺, —PO(O⁻Q⁺)₂, —P(O)(OC₁-C₈alkyl₂)₂, —P(O)(OH₂)₂, —SO₂—OH, —SO₂—C₁-C₈alkyl, —CO—NH—C₁-C₈alkyl, —CONH₂, COO—C₁-C₈alkyl₂, COOH or Si(Me)₃, wherein Q⁺ is H⁺, ammonium or an alkali metal cation;
if k is 2
R₃₀₁ is C₁-C₁₈alkylene, C₃-C₁₈alkenylene or C₃-C₁₈alkinylene, which may be unsubstituted or substituted, by one or more OH, C₁-C₈alkoxy, carboxy, C₁-C₈alkoxycarbonyl; or xylylene; or
R₃₀₁ is a bisacyl radical of an aliphatic dicarboxylic acid having 2 to 36 carbon atoms, or a cycloaliphatic or aromatic dicarboxylic acid having 8-14 carbon atoms;
if k is 3,
R₃₀₁ is a trivalent radical of an aliphatic, cycloaliphatic or aromatic tricarboxylic acid; and
if k is 4, R₃₀₁ is a tetravalent radical of an aliphatic, cycloaliphatic or aromatic tetracarboxylic acid;
and,
(c) other cosmetically acceptable ingredients.

The following examples describe certain embodiments of this invention, but the invention is not limited thereto. It should be understood that numerous changes to the disclosed embodiments could be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated.

The following examples illustrate the invention.

Materials use and their abbreviations.
Monomers: butylacrylate (BA), hydroxyethylacrylate (HEA), hydroxypropylacrylate (HPA) 4-vinyl-pyridine (4-VP).
Modification agents: cyclohexylacid anhydride (CHAA), succinic acid anhydride (SAA), methoxy-poly-ethyleneglycole (MPEG 550-OH), HCl, NaOH.
Solvents: methoxypropylacetate (MPA), Xylene, methoxypropanol (MP), polystyrene (PS), tetrahydrofuran (THF), polyethyleneoxide (POE/PEG).

ATRP process: initiator is 2-bromoethylpropionate (MBP), the catalyst is CuBr/CuBr₂, the ligand is N,N,N′,N″,N″-pentamethyldiethyltriamine (PMDETA). NOR Initiator/regulator is compound O1

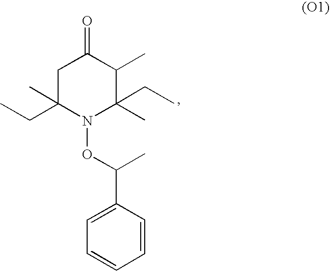

which is prepared according to GB 2335190.

All other materials are commercially available and are used as received.

Some of the solvents used for the synthesis of the instant copolymers may not be suitable for human physiological conditions. Once the synthesis is completed, the solvents can be removed and/or replaced with solvents that are more cosmetically acceptable.

### EXAMPLE 1

### Synthesis of a Linear Polymer Poly(BA)

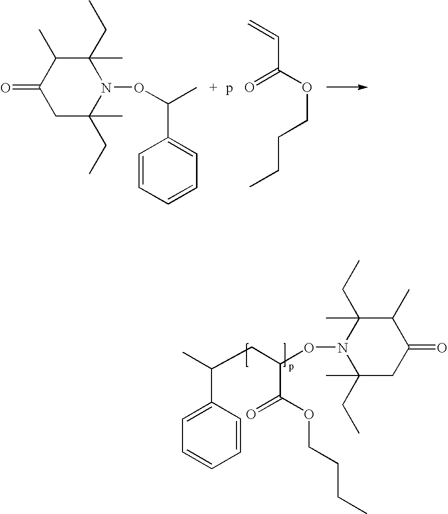

In a 3-necked 1000 ml round bottom flask with magnetic stirring bar, cooler, thermometer, dropping funnel 150.10 g n-Butylacrylate (n-BA, 128.17 g/mol), 8.55 g compound O1 (317.48 g/mol) and 122.13 g of MPA are added, three times degassed with N₂/vacuum and polymerized at 135° C. under N₂ until a conversion of around 8 mol % is reached. 338.89 g of n-BA is slowly added to the reaction with the dropping funnel and polymerized at 135° C. under N₂ until a conversion of around 48 mol %. Residual monomers and solvents are distilled of at 80° C. and 12 mbar.

Yield 47%, GPC (THF, PS-Standard, Mn=7800 g/mol, PD=1.27), liquid.
According to analysis via ¹H-NMR, the degree of polymerization is 75.

### EXAMPLE 2

### Synthesis of a Linear Block Copolymer Poly(n-BA-b-4VP)

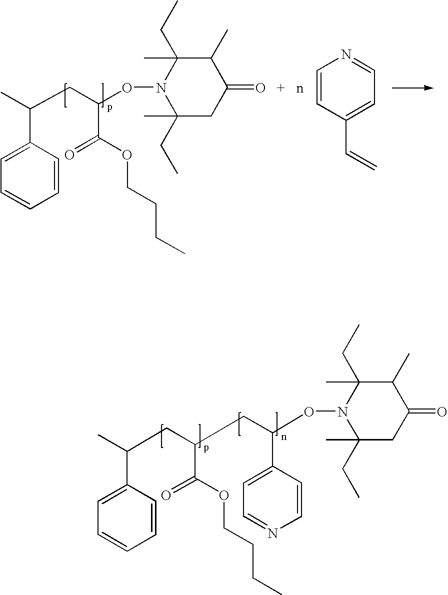

In a 3-necked 500 ml round bottom flask with magnetic stirring bar, cooler, thermometer 214.18 g poly(n-BA) of example 1, 70.90 g 4-vinylpyridine (4-VP, 105.14 g/mol) and 79.70 g of MPA are added, three times degassed with N₂/vacuum and polymerized at 125° C. under N₂ for 8 h. Residual monomers and solvents are distilled off at 80° C. and 12 mbar.

Yield 85%, GPC (THF, PS-Standard, Mn=8600 g/mol, PD=1.24), liquid. According to analysis via ¹H-NMR, the degree of polymerization is: P(BA-b-4VP)=75-b-14.

### EXAMPLE 3

### Poly(n-BA-MPEGA-b-4-VP)

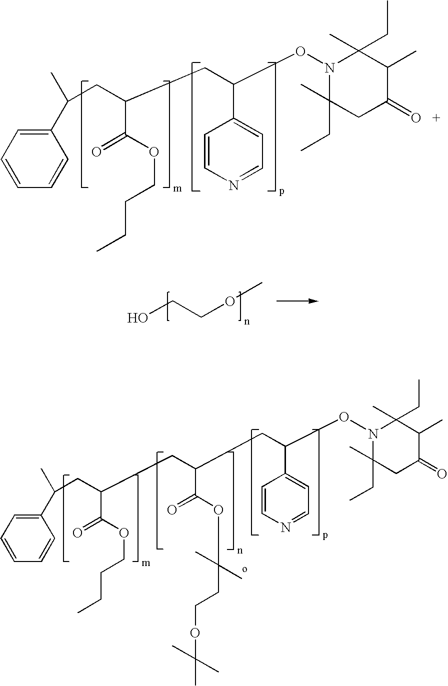

### Transesterification Using MPEG-OH

In a 500 mL flask equipped with a magnetic stirring bar, distillation column with dry ice acetone cooling 92.8 g of Poly(n-BA-b-4-VP) according to example 2 in 107.2 g of Xylene and 114.7 g of MPEG-OH (Mn=550 g/mol) are added and dried by azeotropic distillation of the xylene. Three portions of 0.36 g of tera(isopropyl)orthotitanate are added during 3 h at 190-205° C. The formed n-Butanol is distilled of at low pressure.

187.7 g of Poly(n-BA-MPEGA-b-4-VP) is obtained. Mn=17500 g/mol, PDI=1.6, OH-value=0.05 meq/g. Anaylsis via GPC as well as 1H_NMR indicate almost quantitative conversion of the MPEG-OH.

The resulting polymer is well soluble in water and shows an LCST-type solution behavior (LCST=lower critical solution temperature), i.e. the solubility of the polymer decreases with increasing temperature). A 35 wt % solution of the end product polymer in water is a clear solution at room temperature, but becomes turbid at elevated temperatures above 70° C.

The resulting polymer also formed clear 10 wt % solutions in following organic solvents: butyl acetate, methoxypropylacetate, methoxypropanol, butylglycol and xylene.

### EXAMPLE 4

### Synthesis of a Linear Polymer Poly(BA)

In a 6 liter reactor equipped with stirrer, cooler, thermometer, and monomer feed pumps 1519 g n-Butylacrylate, 209 g compound O1 are added, three times degassed with N₂/vacuum and heated to 115° C. under N₂, where a continuous feed of n-butylacrylate is started over 4 hours and at the same time the reaction mass slowly heated to 135° C. After the end of the monomer feed, the reaction mass is further reacted for 5 h until a solids content of 55% is reached. Afterwards, the non reacted monomer is removed by vacuum distillation.

2812 g of Poly(n-BA) is obtained as liquid polymer, Mn=4554, PDI=1.18 According to analysis via ¹H-NMR, the degree of polymerization is: P(nBA)=35.

### EXAMPLE 5

### Synthesis of a Linear Block Copolymer Poly(n-BA-b-4VP)

In the same reactor as in Example 4, 2674 g of example 4 are loaded together with 1133 g 4-vinylpyridine and heated under N₂ to 135° C. and reacted for 3.5 h until a solids content of 91% is reached. This polymer is used for subsequent transesterifications without further removal of non-reacted 4-vinylpyridine.

3732 g of Polymer P(nBA-b-4VP) is isolated from the reactor, Mn=4779, PDI=1.19 According to analysis via ¹H-NMR, the degree of polymerization is: P(nBA-b-4VP)=35-b-14.

### EXAMPLE 6

### Synthesis of Block Copolymer Poly(n-BA-MPEGA-b-4-VP)

### Transesterification Using MPEG-OH

In the same reactor as in Example 4, 3730 g of the example 5 are loaded together with 3503 g of MPEG-OH (M=550 g/mol) and subjected to vacuum degassing at 130° C. for one hour to remove non-reacted 4-vinylpyridine. 12.0 g of LiOMe-solution (10 wt % lithium methanolate in methanol are added slowly and the transesterification started by distilling off n-butanol at 130° C. and reduced pressure. Additional 5 portions of catalyst are added after every hour: 2×12.0 g and additional 3×14.5 g of LiOMe-solution. After 6 h the reaction is completed by collecting the calculated amount of n-butanol.

6322 g of viscous polymer is obtained; Mn=8829, PDI=1.36
Anaylsis via GPC as well as 1H_NMR indicate almost quantitative conversion of the MPEG-OH.
According to analysis via ¹H-NMR, the degree of polymerization is: P[(nBA-MPEGA)/-b-4VP]=(23-12)-b-14.
OH-number titration: 0.20 meq/g
Amine number titration: 69 mg KOH/g
The 50 wt % solids solution in water displays an LCST of 67° C.
Aside from water, the polymer 6 gives clear solutions 10 wt % in following organic solvents: butyl acetate, methoxypropylacetate, methoxypropanol, butylglycol and xylene. For testing the polymer 6 is dissolved in water to give a clear 50 wt % solids solution.

### EXAMPLE 7

### Synthesis of a Random Copolymer Poly(n-BA-MPEGA)

In the same reactor as in example 4 are loaded 500 g of a poly(n-BA) (Mn=8304, PDI=1.21), which is made analogous to example 4 and 500 g of MPEG-OH (M=550 g/mol). The mixture is heated to 128° C., than 21 g of LiOMe catalysts solution (10 wt % in methanol) are added slowly and n-butanol is slowly distilled off under reduced pressure. Catalyst addition is repeated 5 times each after one hour with 21 g catalyst solution. The transesterification is conducted in total for 6 h until the calculated amount of n-butanol had been distilled off.

918 g of polymer is obtained; Mn=13305, PDI=1.31
Anaylsis via GPC as well as 1H_NMR indicate almost quantitative conversion of the MPEG-OH.
According to analysis via ¹H-NMR, the degree of polymerization is: P(nBA-MPEGA)=(58-19).
The 50 wt % solids solution in water displays an LCST of 70° C.
For testing the polymer 7 is dissolved in water to give a clear 50 wt % solids solution.

### EXAMPLE 8

### Synthesis of a Random Copolymer Poly(n-BA-MPEGA) Comprising Different MPEG-OH: MPEG350, MPEG500, MPEG2000

In a 250 mL flask equipped with a magnetic stirring bar and distillation column are loaded 65 g of a P(nBA) (Mn=8386, PD=1.21; made analog to Example 4), 7.5 g of MPEG-OH (M=350), 7.5 g MPEG-OH (M=500) and 20 g MPEG-OH (M=2000). The mixture is heated to 125° C. and 2 g of LiOMe catalyst solution (10 wt % in MeOH) are slowly added. The transesterification is started by slowly distilling off n-butanol under reduced pressure and increasing the temperature to 130° C. Two additional portions each of 2 g catalyst solution are added after 1 h and 2 h later. After 4 h total reaction time, the transesterification is terminated after the calculated amount of n-butanol is distilled off.

84 g of polymer is obtained; Mn=10490, PDI=1.61
Analysis via GPC as well as ¹H-NMR indicate almost quantitative conversion of the MPEG-OH-mixture.
According to analysis via ¹H-NMR, the degree of polymerization is: P(nBA-MPEGA-mix)=(69-7).

### EXAMPLE 9

### Synthesis of a Random Copolymer Poly(n-BA-MPEGA) Comprising Different MPEG-OH: MPEG350, MPEG500, MPEG5000

In a 250 mL flask equipped with a magnetic stirring bar and distillation column are loaded 65 g of a P(nBA) (Mn=8386, PD=1.21; made analog to Example 4), 7.5 g of MPEG-OH (M=350), 7.5 g MPEG-OH (M=500) and 20 g MPEG-OH (M=5000). The mixture is heated to 125° C. and 2 g of LiOMe catalyst solution (10 wt % in MeOH) are slowly added. The transesterification is started by slowly distilling off n-butanol under reduced pressure and increasing the temperature to 130° C. Two additional portions each of 2 g catalyst solution are added after 1 h and 2 h later. After 4 h total reaction time, the transesterification is terminated after the calculated amount of n-butanol is distilled off.

83 g of polymer is obtained; Mn=9563, PDI=1.75
Analysis via GPC as well as ¹H-NMR indicate almost quantitative conversion of the MPEG-OH-mixture.
According to analysis via ¹H-NMR, the degree of polymerization is: P(nBA-MPEGA-mix)=(71-6).

### EXAMPLE 10

### Synthesis of a Random Copolymer Poly(n-BA-MPEGA-OleA) Comprising Unsaturated Groups

In a 250 mL flask equipped with a magnetic stirring bar and distillation column are loaded 38 g of a P(nBA) (Mn=8386, PD=1.21; made analog to Example 4), 35 g MPEG-OH (M=500) and 27 g oleyl alcohol (techn. grade). The mixture is heated to 125° C. and 2 g of LiOMe catalyst solution (10 wt % in MeOH) are slowly added. The transesterification is started by slowly distilling off n-butanol under reduced pressure and increasing the temperature to 135° C. Two additional portions each of 2 g catalyst solution are added after 1 h and 2 h later. After 4 h total reaction time, the transesterification is terminated after the calculated amount of n-butanol is distilled off.

78 g of liquid polymer is obtained; Mn=13374, PDI=1.87
Analysis via GPC as well as ¹H-NMR indicate almost quantitative conversion of the MPEG-OH and the unsaturated oleyl alcohol.
According to analysis via ¹H-NMR, the degree of polymerization is: P(nBA-MPEGA-OleA)=(32-18-26).

### EXAMPLE 11

### Synthesis of a Non-Polar Block Copolymer Poly[(n-BA-iC12-15A)/-b-4VP]

In a 250 ml flask equipped with a magnetic stirring bar and distillation column are loaded 83.3 g of a 60 wt % MPA-solution of a diblock copolymer P(nBA-b-4VP) (synthesized analogous to example 5; degree of polymerization=76-b-14, Mn=8834, PD=1.27) and 54.1 g of a branched iso-C12-15-alcohol mixture (Lial 125, Condea). After heating the mixture to 125° C., the MPA is distilled under reduced pressure before adding 0.28 g catalyst solution (Ti(AcAc)2(iOPr)₂ Titan-bis-acetylacetonato-bis-isopropylate, 75 wt % in isopropanol). The transesterification is started by slowly distilling off n-butanol under reduced pressure and increasing the temperature to 145° C. Two additional portions each of 0.28 g catalyst solution are added after 1 h and 2 h later. After 4 h total reaction time, the transesterification is terminated after no further n-butanol formation is observed.

76 g of liquid block copolymer is obtained; Mn=12216, PDI=1.27
Analysis via GPC as well as ¹H-NMR indicated almost quantitative conversion of the MPEG-OH and the branched iC12-C15-alcohol.
According to combined analysis of ¹H-NMR and GPC, the degree of polymerization is: P[(nBA-iC12-15A)-b-4VP]=(16-60)-b-14.

### EXAMPLE 12

### Synthesis of a Block Copolymer Poly(n-BA-b-S)

In a 500 ml flask equipped with a magnetic stirring bar and distillation column are loaded 210 g of a P(nBA) (synthesized analog polymer 4; degree of polymerization=76, Mn=8547, PDI=1.19) and 90 g of styrene and are heated under N2 to 125° C. After 5 h the reaction is terminated and the non-reacted styrene is distilled off at reduced pressure.

175 g of block copolymer is obtained; Mn=11828, PDI=1.21
According to analysis of ¹H-NMR the degree of polymerization is: P(nBA-b-S)=(75-b-40).
The resultant very high viscous block copolymer is diluted with MPA to a clear 60 wt % solution.

### EXAMPLE 13

### Synthesis of a Non-Polar Block Copolymer Poly[(n-BA-iC12-15A)-b-S1]

In a 250 ml flask equipped with a magnetic stirring bar and distillation column are loaded 50 g of a 60 wt % MPA-solution of the diblock copolymer of example 12P(nBA-b-S)=75-b-40) and 26.3 g of a branched iso-C12-15-alcohol mixture (Lial 125, Condea). After heating the mixture to 125° C., the MPA is distilled off under reduced pressure before adding 0.15 g catalyst solution (Ti(AcAc)₂(iOPr)₂ Titan-bis-acetylacetonato-bis-isopropylate, 75 wt % in isopropanol). The transesterification is started by slowly distilling off n-butanol under reduced pressure and increasing the temperature to 145° C. Two additional portions each of 0.15 g catalyst solution are added after 2 h and 4 h later. After 6 h total reaction time, the reaction is terminated after no further n-butanol formation is observed.

49 g of liquid block copolymer is obtained; Mn=15072, PDI=1.21
Analysis via GPC as well as ¹H-NMR indicated good conversion of the branched iC12-C15-alcohol.
According to combined analysis of ¹H-NMR and GPC, the degree of polymerization is: P[(nBA-iC12-15A)-b-S=(15-60)-b-40.

### EXAMPLE 14

### Synthesis of a Block Copolymer Poly(n-BA-b-DMAPMA)

In a 500 ml flask equipped with a magnetic stirring bar and distillation column are loaded 150 g of a P(nBA) (synthesized analog polymer 4; degree of polymerization=76, Mn=8547, PDI=1.19) and 150 g of dimethylaminopropyl methacrylamide (DMAPMA) and are heated under N2 to 145° C. After 4.5 h the reaction is terminated and non-reacted monomer DMAPMA is distilled off at high vacuum.

179 g of block copolymer is isolated; Mn=6874, PDI=1.41 (the apparent molecular weight via GPC appeared lower than the starting precursor)
According to analysis of ¹H-NMR the degree of polymerization is: P(nBA-b-DMAPMA)=(75-b-23).
The resultant high viscous block copolymer is diluted with MPA to a clear 60 wt % solution.

### EXAMPLE 15

### Synthesis of a Non-Polar Block Copolymer Poly[(n-BA-BhA)-b-DMAPMA]

In a 250 ml flask equipped with a magnetic stirring bar and distillation column are loaded 41.7 g of a 60 wt % MPA-solution of the diblock copolymer 14 P(nBA-b-DMAPMA)=75-b-23) and 30.0 g of a technical behenyl alcohol (BhOH=linear C16-22-alcohol mixture Nafol 1822 from Condea). After heating the mixture to 125° C., the MPA is distilled off under reduced pressure before adding 0.15 g catalyst solution (Ti(AcAc)₂(iOPr)₂ Titan-bis-acetylacetonato-bis-isopropylate, 75 wt % in isopropanol). The transesterification is started by slowly distilling off n-butanol under reduced pressure and increasing the temperature to 145° C. Two additional portions each of 0.15 g catalyst solution are added after 2 h and 4 h later. After 6 h total reaction time, the reaction is terminated after no further n-butanol formation is observed.

42 g of block copolymer is obtained which solidify at room temperature; Mn=10652, PDI=1.60
Analysis via GPC as well as ¹H-NMR indicated good conversion of the behenyl alcohol.
According to combined analysis of ¹H-NMR and GPC, the degree of polymerization is: P[(nBA-BhA)-b-S=(20-55)-b-23.

### EXAMPLE 16

### Synthesis of a Block Copolymer Poly(n-BA-b-tBA)

In a 500 ml flask equipped with a magnetic stirring bar and distillation column are loaded 123.4 g of a P(nBA) (synthesized analog polymer 1; degree of polymerization=57, Mn=5866, PDI=1.18) and 211.0 g of tert.-butylacrylate (tBA) and are heated under N2 to 120° C. at gentle reflux. After 16 hours, the reaction is terminated when solids content is 53% and non-reacted monomer tBA is distilled off under vacuum.

223 g of block copolymer is isolated; Mn=10052, PDI=1.22
According to analysis of ¹H-NMR the degree of polymerization is: P(nBA-b-tBA)=(57-b-63).

### EXAMPLE 17

### Synthesis of a Non-Polar Block Copolymer Poly[(n-BA-BhA)-b-tBA]

In a 500 ml flask equipped with a magnetic stirring bar and distillation column are loaded 288.7 g of the diblock copolymer 16 (P(nBA-b-tBA)=57/-b-63) and 186.5 g of a technical behenyl alcohol (BhOH=linear C16-22-alcohol mixture Nafol 1822 from Condea). After heating the mixture to 125° C., 0.15 g catalyst solution (Ti(AcAc)₂(iOPr)₂ Titan-bis-acetylacetonato-bis-isopropylate, 75 wt % in isopropanol) are slowly added. The transesterification is started by slowly distilling off n-butanol under reduced pressure and increasing the temperature to 145° C. Two additional portions each of 0.15 g catalyst solution are added after 2 h and 4 h later. After 6 h total reaction time, the reaction is terminated after no further n-butanol formation is observed.

430 g of block copolymer is obtained which solidify at room temperature; Mn=18877, PDI=1.23
Analysis via GPC as well as ¹H-NMR indicated good conversion of the behenyl alcohol.

### EXAMPLE 18

### Synthesis of a Non-Polar Random Copolymer Poly(n-BA-BhA)

In a 250 ml flask equipped with a magnetic stirring bar and distillation column are loaded 85 g of P(nBA) (Mn=8793, PDI=1.20 made analogous to Example 4) and 77 g of a technical behenyl alcohol (BhOH=linear C16-22-alcohol mixture Nafol 1822 from Condea). After heating the mixture to 125° C., 0.43 g catalyst solution (Ti(AcAc)₂(iOPr)₂ Titan-bis-acetylacetonato-bis-isopropylate, 75 wt % in isopropanol) are slowly added. The transesterification is started by slowly distilling off n-butanol under reduced pressure and increasing the temperature to 145° C. Two additional portions each of 0.43 g catalyst solution were added after 2 h and 4 h later. After 6 h total reaction time, the reaction is terminated after no further n-butanol formation is observed.

132 g of block copolymer is isolated which solidify at room temperature; Mn=14811, PDI=1.25
Analysis via GPC indicated good conversion of the behenyl alcohol.

### EXAMPLE 19

### Poly(n-BA-MPEGA-b-4-VP)

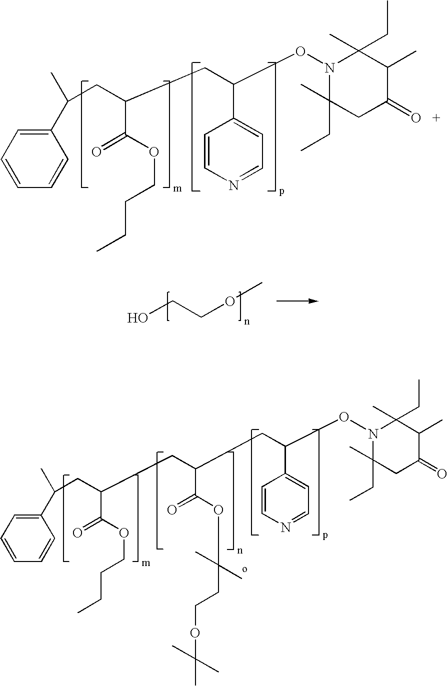

The following substances are polymerized similar to the manner as described in Example 3: 2.27 g compound (O1), 38.22 g n-butylacrylate, 5.84 g vinyl pyridine, and 53.21 g MPEG-OH (Mn=550 g/mol). After completion of the polymerization reaction, all solvents and volatiles are removed by vacuum distillation. A polymeric melt is obtained with a molecular weight of about 15000-20000 Daltons as judged by Gel Permeation Chromotography (GPC). Additionally, a calibrated gas chromatographic analysis of the polymeric melt found that the level of residual 4-vinylpyridine in the melt is less than 2 ppm.

A 50% by weight aqueous solution of the polymer prepared above is prepared.

### EXAMPLE 20

### Sunscreen Composition Preparation

| Part | Trade name | INCI name | Function | % W/W* |
|---|---|---|---|---|
| A | Arlacel 165 | Glyceryl Stearate (and) | Emulsifier, non- | 3.50 |
| | | PEG-100 Stearate | ionic | |
| | Lanette 16 | Cetyl Alcohol | Emulsion | 1.00 |
| | | | Stabilizer | |
| | Cetiol B | Dibutyl Adipate | Emollient/solvent | 5.00 |
| | Cetiol CC | Dicaprylyl Carbonate | Emollient/solvent | 5.00 |
| | Tegosoft DEC | Diethylhexyl Carbonate | Emollient/solvent | 2.00 |
| | Neo Heliopan AV | Octinoxate | Sunscreen Active | 7.50 |
| | Neo Heliopan OS | Octisalate | Sunscreen Active | 2.00 |
| | Parsol 1789 | Avobenzone | Sunscreen Active | 3.00 |
| | Tinosorb ® S | Drug name (proposed): | Sunscreen Active | 1.50 |
| | | Bemotrizinol; | | |
| | | INCI name: Bis- | | |
| | | Ethylhexyloxyphenol | | |
| | | Methoxyphenyl Triazine | | |
| | Orgasol 2002 EXD | Nylon-12 | Spherical | 1.50 |
| | NAT COS | | Particulate to | |
| | | | improve skin feel | |
| B | Water | Water | Diluent/solvent | Qs to 100.00 |
| | Keltrol CG RD | Xanthan Gum | Rheology | 0.22 |
| | | | Modifier | |
| | Glycerin | Glycerin | Humectant | 3.00 |
| | Dissolvine NA2 | Disodium EDTA | Chelating Agent | 0.20 |
| C | Dow Corning 345 | Cyclopentasiloxane | Emollient/Solvent | 2.00 |
| | Fluid | | | |
| | Tinovis ADM | Sodium Acrylates | Rheology | 0.83 |
| | | Copolymer (and) Mineral | Modifier | |
| | | Oil (and) PPG-1 Trideceth-6 | | |
| | Tinosorb ® M | Drug name (proposed): | Sunscreen Active | 3.00 (as is) |
| | | Bisoctrizole; | | or |
| | | INCI name: Methylene Bis- | | 1.50 (active |
| | | Benzotriazolyl | | level) |
| | | Tetramethylbutylphenol | | |
| | Phenonip | Phenoxyethanol (and) | Preservative | 1.00 |
| | | Methylparaben (and) | | |
| | | Ethylparaben (and) | | |
| | | Butylparaben (and) | | |
| | | Propylparaben (and) | | |
| | | Isobutylparaben | | |
| | Sodium Hydroxide | Sodium Hydroxide (15% | pH adjustor | Qs to pH ~5.3-6.1 |
| | | aqueous solution) | | |

*The components are added to the sunscreen composition on a % weight/weight of component (as active) based on the weight of the total composition.

Combine the ingredients of part A. Heat up part A to 80° C. with mixing. Mix until uniform, and add Nylon-12 with moderate agitation.

Prepare part B: first, disperse Xanthan Gum into the water and heat up to 80° C. When uniform, add the rest of part B one by one, mix until uniform.

Add part A into part B under stirring, and then homogenize with an Ultra Turrax pos 2 for 40 sec/100 g.

Cool down under stirring, to 40° C. and add the ingredients of part C one by one in the given order. Mix until uniform. If necessary, adjust pH with aqueous solution of Sodium Hydroxide to 5.3-6.1

### EXAMPLE 21

### Testing Protocol

The test protocol described below is used to mimic the application of the sunscreen composition to human skin and test the initial SPF and the SPF after eighty minutes of water exposure of the instant compositions.

The following laboratory equipment is used:

VITRO-SKIN® N-19, Foam block, Hydration Chamber, Powder Free Rubber Finger Cots and Glassless slide mounts are obtained from IMS, Inc. (70 Robinson Blvd, Orange, Conn., USA);
Water bath (#05-719-7F), Corning Hotplate Stirrer (#11-497-8A), Calfamo Compact Digital Stirrer (#14-500-7), Glycerol Aqueous Solution (#AC277366-0010) are obtained from Fisher Scientific Catalog; and

Optometrics SPF 290 is obtained from Optometrics LLC. (8 Nemco Way, Stony Brook Industrial Park, Ayer, Mass., USA).

An aqueous solution of glycerin (300 g of 14.7% by weight) is prepared and poured on the bottom of the hydration chamber. The shelves are placed in the chamber that is covered with a lid. VITRO-SKIN substrate is cut into 4.1 cm×4.1 cm pieces that are placed on the shelves in a hydration chamber and hydrated for 16-22 hours prior to the tests.

Optometrics SPF 290S is turned on followed by the manufacturer's directions for instrument calibration, blank and sample measurements.

A piece of substrate is placed in a slide mount and used as a reference for the in vitro SPF measurements. Another piece of substrate is placed on a plastic-covered foam block and product application is made to the “topography” side of the substrate (the rough side). The test composition (0.033 g) is applied evenly across a 4 cm×4 cm section of the substrate, which results in an application dose of 2 mg/sq. cm and rubbed into the substrate with a finger covered with finger cot. After this, the substrate is placed on a slide mount.

The in vitro SPF measurements are made both prior to and after sample immersion in water with stirring for 80 minutes at a water temperature of 37+/−0.5° C. All initial measurements are made after the 15 minute dry-down period. After water exposure, the samples are removed, air-dried for about 30 minutes, placed back in the controlled humidity chamber for 120 minutes followed by the 15 minute dry-down period. The reference slides are immersed in the water bath for the same amount of time. An Optometrics SPF 290S is used to determine UV absorbance for each formulation in the 290-400 nm wavelength range. A minimum of three consecutive measurements on three separate areas of the slide are conducted. SPF, UVA/UVB and Critical Wavelength in vitro values for each sample—before and after water immersion are recorded. The % SPF remaining after eighty minute exposure to water is calculated by:

(a/b)×100=%SPF remaining

(a) is SPF value after 80 minutes of water exposure and (b) is initial SPF value.

### EXAMPLE 22

### Sunscreen Composition Testing for Water Resistant Properties

The base sunscreen composition of Instant Example 20 is formulated with the copolymer of Instant Example 19 and compared with other commercially available polymers and copolymers. The composition of Instant Example 20 is prepared individually with the specified amount of each test polymer or copolymer. Commercially available polymers were added to the oil phase or water phase of the formulation, or post-added according to the recommendations described in the manufacturer's literature.

Each sunscreen formulation is evaluated according to the protocol of Instant Example 21. The experimental results are given below.

| Polymer Tested | wt/wt %* | % SPF remaining |
|---|---|---|
| None | 0 | 3.1 |
| Cosmedia DC | 3 | 4.5 |
| Polycrylene | 3 | 5.7 |
| DC FA 4001 CM | 3 | 6.0 |
| Silicone Acrylate | | |
| Ganex V-220 | 3 | 6.8 |
| DC FA 4002 ID | 3 | 7.4 |
| Silicone Acrylate | | |
| Phospholipon 90H | 3 | 35.0 |
| Dermacryl AQF | 3 | 40.5 |
| Ganex WP-660 | 3 | 53.0 |
| Stantiv OMA-2 | 3 | 53.3 |
| Dermacryl-79 | 3 | 55.9 |
| Instant Example 19 | 1 | 63.5 |

*The commercially available polymers are added to the sunscreen composition at a 3% weight/weight of component (as active) based on the weight of the total composition.

Instant Example 19 is added at a 1% weight/weight of component (as active) based on the weight of the total composition.

Cosmedia DC is a hydrogenated dimer Dilinoleyl/Dimethylcarbonate Copolymer and is obtained from Cognis.

Polycrylene is Polyester-8 which is a copolymer of adipic acid (q.v.) and neopentyl glycol (q.v.) end-capped with either octyldodecanol (q.v.) or a cyanodiphenylpropenoyl group and is obtained from RTD Hall Star.

DC FA 4001 CM Silicone Acrylate is a copolymer of polytrimethylsiloxymethacrylate and one or more monomers consisting of acrylic acid, methacrylic acid, or one of their simple esters dissolved in cyclopentasiloxane and is obtained from Dow Corning.

Ganex V-220 is a copolymer of vinylpyrrolidone and eicosene and is obtained from ISP.

DC FA 4002 ID Silicone Acrylate is a copolymer of polytrimethylsiloxymethacrylate and one or more monomers consisting of acrylic acid, methacrylic acid, or one of their simple esters dissolved in isododecane and is obtained from Dow Corning.

Phospholipon 90H is hydrogenated lecithin and is obtained from Phospholipid GmbH.

Dermacryl AQF is a copolymer of acrylates and is obtained from National Starch and Chemical Company.

Ganex WP-660 is a copolymer of vinyl pyrrolidone and 1-triacontane and is obtained from ISP.

Stantiv OMA-2 is a linear copolymer of maleic anhydride and octadecene and is dissolved a mixture of methyl acetyl ricinoleate and dimethylheptyl adipate.

Dermacryl-79 is a copolymer of octylacrylamide and one or more monomers consisting of acrylic acid, methacrylic acid or one of their simple esters and is obtained from National Starch and Chemical Company.

The data demonstrate the instant copolymer provide excellent water proofing properties in sunscreen compositions at one-third of the concentration when compared to other polymers and copolymers of the prior art and commerce.

### EXAMPLE 23

### Sunscreen Composition Testing for Water Resistant Properties

A commercial sunscreen formulation (Cetaphil SPF 15, Galderma) is obtained and is thoroughly mixed individually with the specified amount of each test polymer or copolymer. Each sunscreen formulation is evaluated according to the protocol of Instant Example 21. The experimental results are given below.

| Polymer Tested | wt/wt %* | % SPF remaining |
|---|---|---|
| None | 0 | 10.1 |
| Dermacryl AQF | 2 | 9.1 |
| Allianz OPT | 1 | 49.1 |
| Instant Example 19 | 1 | 81.8 |

*The polymers are added to the sunscreen composition on a % weight/weight of component (as active) based on the weight of the total composition.

Dermacryl AQF is a copolymer of acrylates and is obtained from National Starch and Chemical Company.

Allianz OPT is a copolymer of: methacrylic acid, methyl methacrylate, butyl acrylate, and cetyl-eicosinyl methacrylate and is obtained from ISP.

Cetaphil SPF 15 is a commercial sunscreen formulation that contains sunscreen actives: Avobenzone 3%; Octocrylene 10%; and

### Inactive Ingredients (Function):

Water (solvent),
Isopropyl adipate (emollient, solvent),
Cyclomethicone (emollient, solvent),
Glyceryl Stearate (and) PEG-100 Stearate (emulsifier, non-ionic),
Glycerin (humectant),
Polymethyl Metacrylate (spherical particulate to improve the skin feel,
Phenoxyethanol (preservative),
Benzyl Alcohol (preservative),
Acrylates/C10-30 Alkyl Acrylate Crosspolymer (polymeric emulsifier, rheology modifier),
Tocopheryl Acetate (antioxidant),
Carbomer (rheology modifier),
Disodium EDTA (chelating agent), and
Triethanolamine (pH adjustor).

The data demonstrate the instant terpolymer provide excellent water proofing properties in sunscreen compositions when compared to other polymers and copolymers of the prior art and commerce.

### EXAMPLE 24

### Very Water Resistant Properties of the Sunscreen Formulations

The water resistant properties of the instant copolymers are studied according to: the FDA Final Monograph “Evaluation of Sunscreen Efficacy—Sun Protection Factor (SPF) Assay and Very Water Resistant Assay” (in vivo). The instant copolymers are studied at 1% w/w based on solids and the data obtained from the in vivo evaluation of the very water resistant properties of the sunscreen formulations containing the instant copolymers is given below.

| | | Final SPF | Per Cent SPF |
|---|---|---|---|
| Sample | Initial SPF | After Exposure | After Exposure |
| Control* | 15.26 | 7.46 | 48 |
| Instant 19 | 15.34 | 10.94 | 71 |

*A commercial daily moisturizing sunscreen formulation (Cetaphil SPF 15, Lot 049957, Galderma) is obtained and is thoroughly mixed with the specified amount of each instant terpolymer.

It is found that, at concentration 1% w/w based on solids, the instant copolymers provide a significant improvement of very water-resistant properties of a sunscreen formulation.

### EXAMPLE 25

### Contact Angles of Water and Surface Properties

A test methodology that utilizes measurements of the contact angle of water to quantify the effects on the surface properties of a skin-substitute substrate is employed. This methodology is used as an effective tool for optimizing product development, differentiating among skin care products, competitive benchmarking, and screening of the polymers. It is described in the article entitled “Correlating Water Contact Angles and Moisturization/Sensory Claims” by Olga V. Dueva-Koganov, Scott Jaynes, Colleen Rocafort, Shaun Barker and Jianwen Mao—Cosmetics & Toiletries, January 2007, Vol. 122, No. 1, pp. 20-27. The data presented in the graph of this article shows that contact angle measurements can be used to quantify and compare the effects of skin care products on the surface properties of a skin-like substrate and is presented in tabular form below. Products that generate relatively low contact angles tend to make more sensory claims related to light and non-greasy feel, while products that produce relatively high contact angles tend to make more claims related to long-term moisturization.

| Contact | Products | Products | Products |
|---|---|---|---|
| Angles | % in A* | % in B** | % in C*** |
| 40-50 | 100 | 50 | 0 |
| 50-60 | 100 | 40 | 20 |
| 60-70 | 60 | 30 | 60 |
| 70-80 | 70 | 15 | 70 |
| 80-90 | 50 | 0 | 100 |

*A = Products that are light and/or nongreasy.
**B = Products that provide 8-12 hour moisturization.
***C = Products that provide 24 hour moisturization.

### EXAMPLE 26

### Measurement of Contact Angles after Application of the Instant Copolymers

Contact angles are measured instrumentally according to the static or sessile drop method and using deionized water as a probe solution and VITRO SKIN that mimics the surface properties of human skin as a substrate. A piece of hydrated substrate is mounted in a glassless slide and air-dried in a flat position with application side up for 15 minutes. It is used as a reference for untreated substrate during the contact angle measurements. Exactly 0.032 g of aqueous solutions or dispersions of test polymers are applied evenly across a 4 cm×4 cm section of the substrate (on the “skin topography” side). Immediately after product application, the product is rubbed into the substrate with a finger covered with fingercot. After that the substrate is placed in a slide mount and air-dried for 15 minutes. Before measurements, substrate is removed from the slide mount and cut to several small pieces, which are used for the measurements. The use of small size piece is necessary to assure its flat position on the sample table. Extra care is taken to ensure that the rough side is up and the film is flat. Contact angle measurements are conducted expeditiously—within approximately 1 minute. Controlled humidity conditions are utilized.

### Materials

### DSA-10 Contact Angle Measuring System, Kruss Gmb.

### VITRO SKIN (N-19), IMS Inc.,

Powder Free Rubber Finger Cots (#11-392-9B) are available from the Fisher Scientific Catalog.
Instant terpolymers and competitive water-resistant polymers Allianz OPT (ISP) and Dermacryl AQF (National Starch) are evaluated according to the methodology described above.

### Contact Angles after Application of the Polymers

| | | Contact Angle, |
|---|---|---|
| Polymer Tested | wt/wt %* | Theta (M) deg. |
| Control (Vitro Skin) | None | 103.6 |
| Instant Example 19 | 5 | 44.8 |
| Instant Example 19 | 2.5 | 40.0 |
| Instant Example 19 | 1 | 41.9 |
| Instant Example 19 | 0.5 | 38.3 |
| Instant Example 19 | 0.25 | 49.5 |
| Allianz OPT | 5 | 109.8 |
| Allianz OPT | 2.5 | 108.6 |
| Allianz OPT | 1 | 111.9 |
| Allianz OPT | 0.5 | 99.7 |
| Allianz OPT | 0.25 | 104.8 |
| Dermacryl AQF | 5 | 116.1 |
| Dermacryl AQF | 2.5 | 117.3 |

*Indicates aqueous solutions of the test sample as per cent polymer solids based on the total weight of the solution.

The instant copolymers and competitive water-resistant polymers demonstrate strong differences in their effects on the surface properties of VITRO SKIN. The results presented in the table above indicate that the instant copolymers can potentially contribute to light skin feel—a desirable characteristic for water resistant polymers. On the contrary—the competitive benchmarks (Allianz OPT and Dermacryl AQF) generate primarily a hydrophobic modification of the substrate and are less likely to produce light skin feel.

What is claimed:

1. A sunscreen composition comprising
(a) at least one UV screening agent;
(b) at least one select copolymer 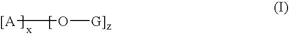
wherein
x and z represent the percentage by weight that each repeating unit or derived monomer is contained within the copolymer;
x and z add up to total 100 weight percent relative to the total weight of the copolymer;
x and z refer to repeating units;
z is from about 0.001% to about 99.999% by weight of the copolymer;
x is from about 0.001% to about 99.999% by weight of the copolymer;
A is a polymer;
G is covalently bonded to the polymer A through an oxygen linking group;
O is an oxygen atom;
G is 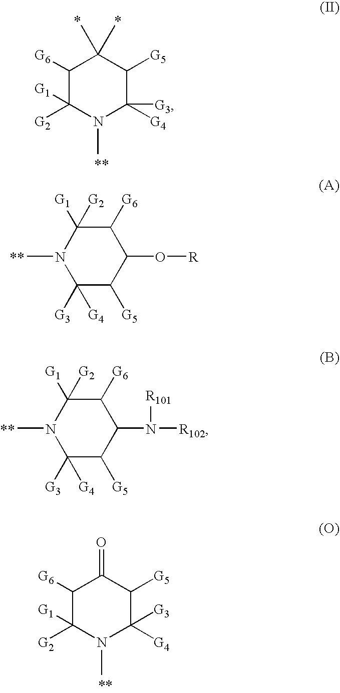
wherein
G₁, G₂, G₃, G₄ are independently C₁-C₆alkyl or G₁ and G₂ or G₃ and G₄, or G₁ and G₂ and
G₃ and G₄ together form a C₅-C₁₂cycloalkyl group;
G₅, G₆ independently are H, C₁-C₁₈alkyl, phenyl, naphthyl or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, of a cycloaliphatic carboxylic acid having 7 to 15 carbon atoms, or an alpha, beta-unsaturated carboxylic acid having 3 to 5 carbon atoms or of an aromatic carboxylic acid having 7 to 15 carbon atoms;
R₁₀₁, is C₁-C₁₂alkyl, C₅-C₇cycloalkyl, C₇-C₈aralkyl, C₂-C₁₈alkanoyl, C₃-C₅alkenoyl or benzoyl;
R¹⁰² is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or
G is 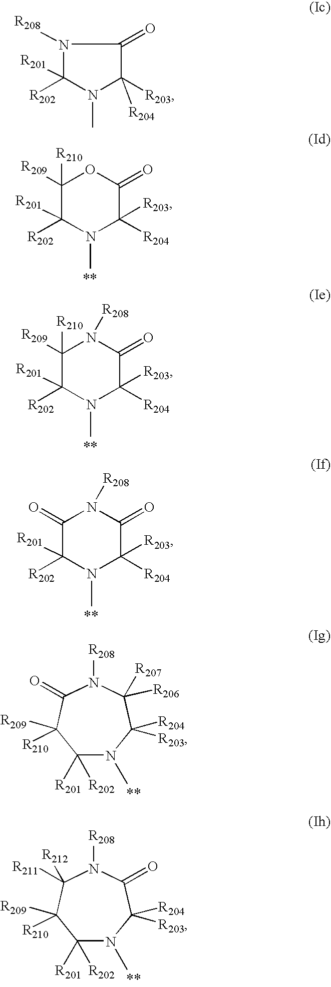
wherein R₂₀₁, R₂₀₂, R₂₀₃ and R₂₀₄ independently of each other are C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl or R₂₀₁ and R₂₀₂ and/or R₂₀₃ and R₂₀₄ together with the linking carbon atom form a C₃-C₁₂cycloalkyl radical;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by one or more OH, halogen or a group—O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉-phenylalkyl, C₅-C₁₀heteroaryl, —C(O)—C₁-C₁₈alkyl, —O—C₁-C₁₈alkyl or —COOC₁-C₁₈alkyl;
R₂₀₉, R₂₁₀, R₂₁₁ and R₂₁₂ are independently hydrogen, phenyl or C₁-C₁₈alkyl; or
G is 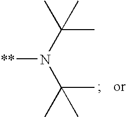
G is 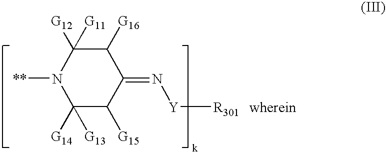
G₁₁, G₁₂, G₁₃ and G₁₄ are independently C₁-C₄alkyl or G₁₁ and G₁₂ together and G₁₃ and
G₁₄ together, or G₁₁ and G₁₂ together or G₁₃ and G₁₄ together are pentamethylene;
G₁₅ and G₁₆ are each independently of the other hydrogen or C₁-C₄alkyl;
X is as defined above;
k is 1, 2, 3, or 4
Y is O or NR₃₀₂ or when k is 1 and R₃₀, represents alkyl or aryl Y is additionally a direct bond;
R₃₀₂ is H, C₁-C₁₈alkyl or phenyl;
if k is 1
R₃₀₁ is H, straight or branched C₁-C₁₈alkyl, C₃-C₁₈alkenyl or C₃-C₁₈alkinyl, which may be unsubstituted or substituted, by one or more OH, C₁-C₈alkoxy, carboxy, C₁-C₈alkoxycarbonyl;
C₅-C₁₂cycloalkyl or C₅-C₁₂cycloalkenyl;
phenyl, C₇-C₉-phenylalkyl or naphthyl which may be unsubstituted or substituted by one or more C₁-C₈alkyl, halogen, OH, C₁-C₈alkoxy, carboxy, C₁-C₈alkoxycarbonyl;
—C(O)—C₁-C₃₆alkyl, or an acyl moiety of a □,□-unsaturated carboxylic acid having 3 to 5 carbon atoms or of an aromatic carboxylic acid having 7 to 15 carbon atoms;
—SO₃⁻Q⁺, —PO(O⁻Q⁺)₂, —P(O)(OC₁-C₈alkyl₂)₂, —P(O)(OH₂)₂, —SO₂—OH, —SO₂—C₁-C₈alkyl, —CO—NH—C₁-C₈alkyl, —CONH₂, COO—C₁-C₈alkyl₂, COOH or Si(Me)₃, wherein Q⁺ is H⁺, ammonium or an alkali metal cation;
if k is 2
R₃₀₁ is C₁-C₁₈alkylene, C₃-C₁₈alkenylene or C₃-C₁₈alkinylene, which may be unsubstituted or substituted, by one or more OH, C₁-C₈alkoxy, carboxy, C₁-C₈alkoxycarbonyl; or xylylene; or
R₃₀₁ is a bisacyl radical of an aliphatic dicarboxylic acid having 2 to 36 carbon atoms, or a cycloaliphatic or aromatic dicarboxylic acid having 8-14 carbon atoms;
if k is 3,
R₃₀₁ is a trivalent radical of an aliphatic, cycloaliphatic or aromatic tricarboxylic acid; and
if k is 4, R₃₀₁ is a tetravalent radical of an aliphatic, cycloaliphatic or aromatic tetracarboxylic acid;
and, optionally
(c) other cosmetically acceptable ingredients.

2. A composition according to claim 1 wherein for component b) formula (I) G is 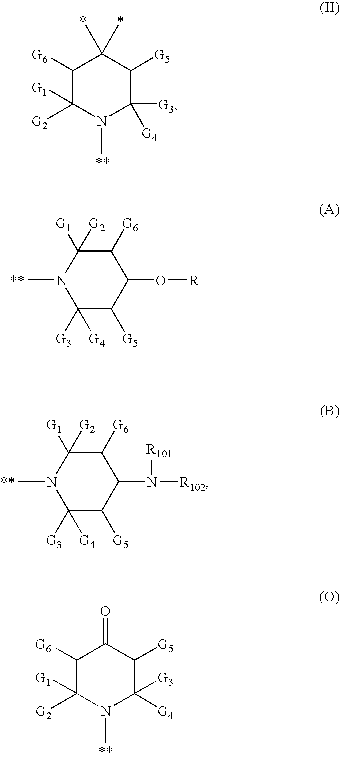
wherein
G₁, G₂, G₃, G₄ are independently C₁-C₁alkyl or G₁ and G₂ or G₃ and G₄, or G₁ and G₂ and G₃ and G₄ together form a C₅-C₁₀cycloalkyl group;
G₅, G₆ independently are H, C₁-C₁₈alkyl, phenyl, or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, or of a cycloaliphatic carboxylic acid having 7 to 15 carbon atoms;
R₁₀₁ is C₁-C₁₂alkyl, C₅-C₇cycloalkyl, C₇-C₈aralkyl, C₂-C₁₈alkanoyl, C₃-C₅alkenoyl or benzoyl;
R₁₀₂ is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or
G is 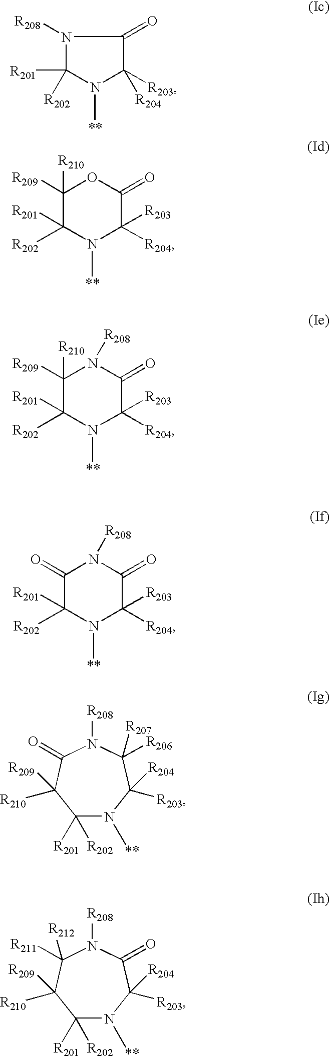
wherein R₂₀₁, R₂₀₂, R₂₀₃ and R₂₀₄ independently of each other are C₁-C₆alkyl, C₃-C₁₈alkenyl; C₁-C₁₈alkyl, or C₃-C₁₈alkenyl, which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl; C₁-C₁₈alkyl which are substituted by one or more OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉phenylalkyl, C₅-C₁₀heteroaryl, —C(O)—C₁-C₁₈alkyl, —O—C₁-C₁₈alkyl or —COOC₁-C₁₈alkyl;
R₂₀₉, R₂₁₀, R₂₁₁ and R₂₁₂ are independently hydrogen, phenyl or C₁-C₁₈alkyl; or
G is 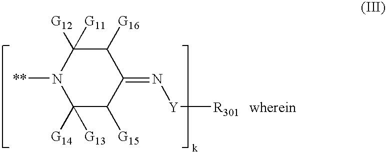
G₁₁, G₁₂, G₁₃ and G₁₄ are independently C₁-C₄alkyl or G₁₁ and G₁₂ together and G₁₃ and
G₁₄ together, or G₁₁ and G₁₂ together or G₁₃ and G₁₄ together are pentamethylene;
G₁₅ and G₁₆ are each independently of the other hydrogen or C₁-C₄alkyl;
k is 1
Y is O or NR₃₀₂ or Y is additionally a direct bond;
R₃₀₂ is H, C₁-C₁₈alkyl or phenyl;
if k is 1
R₃₀₁ is H, straight or branched C₁-C₁₈alkyl, C₃-C₁₈alkenyl or C₃-C₁₈alkinyl, which may be unsubstituted or substituted, by one or more OH, C₁-C₈alkoxy, carboxy, C₁-C₈alkoxycarbonyl;
C₅-C₁₂cycloalkyl or C₅-C₁₂cycloalkenyl; C₇-C₉-phenylalkyl which may be unsubstituted or substituted by one or more C₁-C₈alkyl, halogen, OH, C₁-C₈alkoxy, carboxy, C₁-C₈alkoxycarbonyl; —C(O)—C₁-C₃₆alkyl;
—SO₃⁻Q⁺, —PO(O⁻Q⁺)₂, —P(O)(OC₁-C₈alkyl₂)₂, —P(O)(OH₂)₂, —SO₂—OH, —SO₂—C₁-C₈alkyl, —CO—NH—C₁-C₈alkyl, —CONH₂, COO—C₁-C₈alkyl₂, COOH or Si(Me)₃, wherein Q⁺ is H⁺, ammonium or an alkali metal cation.

3. A composition according to claim 1 wherein for component b) formula (I) G is 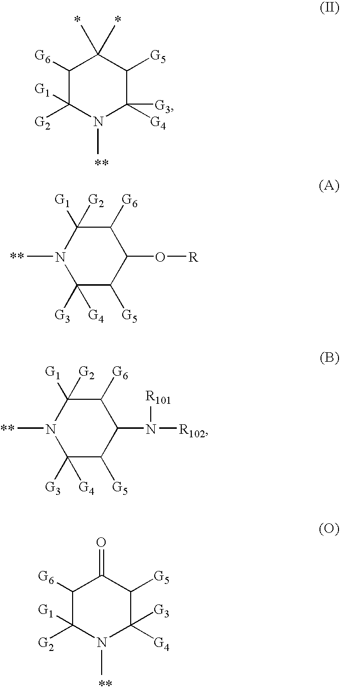
wherein
G₁, G₂, G₃, G₄ are independently C₁-C₁alkyl or G₁ and G₂ or G₃ and G₄, or G₁ and G₂ and G₃ and G₄ together form a C₅-C₁₀cycloalkyl group;
G₅, G₆ independently are H, C₁-C₁₈alkyl, phenyl, or a group COOC₁-C₁₈alkyl;
*denotes a valence and **denotes point of attachment to said polymer A;
R is hydrogen, C₁-C₁₈alkyl which is uninterrupted or interrupted by one or more oxygen atoms, cyanoethyl, benzoyl, glycidyl, a monovalent radical of an aliphatic carboxylic acid having 2 to 18 carbon atoms, or of a cycloaliphatic carboxylic acid having 7 to 15 carbon atoms;
R₁₀₁ is C₁-C₁₂alkyl, C₅-C₇cycloalkyl, C₇-C₈aralkyl, C₂-C₁₈alkanoyl, C₃-C₅alkenoyl or benzoyl;
R₁₀₂ is C₁-C₁₈alkyl, C₅-C₇cycloalkyl, C₂-C₈alkenyl unsubstituted or substituted by a cyano, carbonyl or carbamide group, or is glycidyl, a group of the formula —CH₂CH(OH)-Z or of the formula —CO-Z or —CONH-Z wherein Z is hydrogen, methyl or phenyl; or
G is 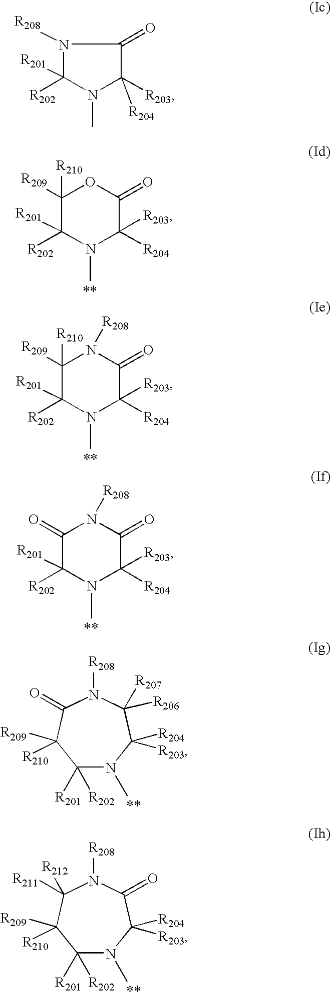
wherein R₂₀₁, R₂₀₂, R₂₀₃ and R₂₀₄ independently of each other are C₁-C₆alkyl, C₃-C₁₈alkenyl; C₁-C₁₈alkyl, or C₃-C₁₈alkenyl, which are substituted by OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl;
R₂₀₅, R₂₀₆ and R₂₀₇ independently are hydrogen, C₁-C₁₈alkyl or C₆-C₁₀aryl;
R₂₀₈ is hydrogen, OH, C₁-C₁₈alkyl; C₁-C₁₈alkyl which are substituted by one or more OH, halogen or a group —O—C(O)—R₂₀₅, C₂-C₁₈alkyl which is interrupted by at least one O atom and/or NR₂₀₅ group, C₃-C₁₂cycloalkyl or C₆-C₁₀aryl, C₇-C₉phenylalkyl, C₅-C₁₀heteroaryl, —C(O)—C₁-C₁₈alkyl, —O—C₁-C₁₈alkyl or —COOC₁-C₁₈alkyl; and
R₂₀₉, R₂₁₀, R₂₁₁ and R₂₁₂ are independently hydrogen, phenyl or C₁-C₁₈alkyl.

4. A composition according to claim 1 wherein for component b formula (I) z is from about 0.001% to about 80% by weight of the copolymer and x is from about 20% to about 99.999% by weight of the copolymer.

5. A composition according to claim 4 wherein for component b formula (I) z is from about 0.001% to about 40% by weight of the copolymer and x is from about 60% to about 99.999% by weight of the copolymer.

6. A composition according to claim 5 wherein for component b formula (I) z is from about 0.001% to about 20% by weight of the copolymer and x is from about 80% to about 99.999% by weight of the copolymer.

7. A composition according to claim 6 wherein for component b formula (I) z is from about 0.001% to about 10% by weight of the copolymer and x is from about 90% to about 99.999% by weight of the copolymer.

8. A composition according to claim 1 wherein polymer A of component b) formula (I) is derived from monomers that are selected from the group consisting of anionic water soluble monomers, nonionic water soluble monomers, cationic water soluble monomers and water insoluble monomers.

9. A composition according to claim 1 wherein polymer A of component b) formula (I) possesses a structure that is selected from the group consisting of copolymer, terpolymer, block, star, random, linear, branched, crosslinked and not crosslinked.

10. A composition according to claim 1 wherein component b) formula (I) has a weight average molecular weight of from about 500 to about 1 million Daltons.

11. A composition according to claim 10 wherein component b) formula (I) has a weight average molecular weight of from about 500 to about 500,000 Daltons.

12. A composition according to claim 11 wherein component b) formula (I) has a weight average molecular weight of from about 500 to about 100,000 Daltons.

13. A composition according to claim 12 wherein component b) formula (I) has a weight average molecular weight of from about 1000 to about 75,000 Daltons.

14. A composition according to claim 1 wherein component b) formula (I) is present in an amount from about 0.01 weight percent to about 50 weight percent based on the weight of the total composition.

15. A composition according to claim 14 wherein component b) formula (I) is present in an amount from about 0.1 weight percent to about 25 weight percent based on the weight of the total composition.

16. A composition according to claim 15 wherein component b) formula (I) is present in an amount from about 0.1 weight percent to about 10 weight percent based on the weight of the total composition.

17. A composition according to claim 1 wherein component a) comprises at least one UV screening agent selected from the group consisting of organic sunscreens, inorganic sunscreens and mixtures thereof.

18. A composition according to claim 17 wherein said inorganic sunscreen is selected from the group consisting of titanium oxide, iron oxide, zinc oxide, zirconium oxide, cerium oxide and mixtures thereof.

19. A composition according to claim 17 wherein said organic sunscreen is selected from the group consisting of triazines, benzotriazoles, benzophenones, vinyl group-containing amides, cinnamic acid amides, sulfonated benzimidazoles and mixtures thereof.

20. A composition according to claim 17 wherein component a) is present in micronized or non-micronized form.

21. A composition according to claim 1 wherein component a) is present in an amount from about 0.1 weight percent to about 30 weight percent based on the weight of the total composition.

22. A composition according to claim 21 wherein component a) is present in an amount from about 1 weight percent to about 20 weight percent based on the weight of the total composition.

23. A composition according to claim 22 wherein component a) is present in an amount from about 1 weight percent to about 5 weight percent based on the weight of the total composition.

24. A method of increasing the sun protection factor of a sunscreen composition wherein said method comprises incorporating into said composition an effective amount of at least one select copolymer according to formula (I) of claim 1.

25. A method of improved UV protection of mammalian hair and/or skin from the damaging effects of UV radiation wherein said method comprises applying to said skin and/or said hair an effective amount of a sunscreen composition according to claim 1.
